(19) United States
(12) Patent Application Publication
(10) Pub. No.: US 2023/0027906 A1
(43) Pub. Date: Jan. 26, 2023

(54) TERT Promoter Droplet Digital PCR Assay for the Diagnosis of Malignant Cancers

(71) Applicant: The General Hospital Corporation, Boston, MA (US)

(72) Inventors: Leonora Balaj, Lincoln, MA (US); Koushik Muralidharan, Boston, MA (US); Anudeep Yekula, Boston, MA (US); Bob S. Carter, Boston, MA (US)

(21) Appl. No.: 17/781,423

(22) PCT Filed: Dec. 7, 2020
(86) PCT No.: PCT/US2020/063663

§ 371 Date: Jun. 1, 2022

Related U.S. Application Data
(60) Provisional application No. 63/028,407, filed on May 21, 2020.
(60) Provisional application No. 62/944,922, filed on Dec. 6, 2019.

(51) Int. Cl. C12Q 1/6886

(52) U.S. Cl. CPC C12Q 1/6886; C12Q 1/6858

(57) ABSTRACT

Aspects of the present disclosure relate to methods for detecting mutant TERT promoter sequences (e.g., C228T, C250T) that provide several improvements over conventional detection methods, thereby enabling detection of such mutations in biological fluid samples (e.g., plasma), which contain miniscule amounts of nucleic acids. Such improvements include, but are not limited to, improvements in detection sensitivity and specificity, which allows detection of mutations in a biological sample having a low level of nucleic acids such as a plasma sample from a patient having brain cancer (e.g., glioma).

### CLAIM OF PRIORITY

This application claims the benefit of U.S. Provisional Patent Application No. 62/944,922, filed on Dec. 6, 2019, and U.S. Provisional Patent Application No. 63/028,407, filed on May 21, 2020, each of which is incorporated by reference herein in its entirety.

### FEDERALLY SPONSORED RESEARCH OR DEVELOPMENT

This invention was made with government support under Grant Nos. CA069246 and CA230697 awarded by the National Institutes of Health. The government has certain rights in the invention.

### FIELD OF THE INVENTION

The subject matter disclosed herein generally relates to detection of telomerase reverse transcriptase (TERT) promoter mutations by liquid biopsy.

### BACKGROUND OF THE INVENTION

Liquid biopsy for the detection and monitoring of brain tumors is of significant clinical interest. Upon clinical presentation, patients typically undergo imaging followed by biopsy alone or biopsy with resection for diagnosis and determination of histopathological classification. While tissue biopsy is invasive and can, in some cases, be high risk, liquid biopsy can offer a less invasive sampling approach that still affords significant clinical information for diagnosis and treatment. In addition, liquid biopsy can be performed more frequently to allow for longitudinal treatment monitoring. The detection of glioma mutations via liquid biopsy of cerebrospinal fluid has been demonstrated; however, the ability to detect mutations in cell free DNA (cfDNA) in plasma with similar sensitivities has been limited.

### SUMMARY OF THE INVENTION

The present disclosure is based, at least in part, on the development of methods for detecting mutations in the promoter region of the telomerase reverse transcriptase (TERT) gene that provide several improvements over conventional detection methods. Such improvements include, but are not limited to, improvements in detection sensitivity and specificity, which allows detection of mutations in a biological sample having a low level of nucleic acids such as a plasma sample from a patient having brain cancer (e.g., glioma).

Accordingly, aspects of the present disclosure provide a method for detecting mutations in a telomerase reverse transcriptase (TERT) promoter sequence, the method comprising incubating, in a reaction mixture, a DNA sample comprising the TERT promoter sequence, wherein the DNA sample comprises cell-free DNA (cfDNA) and exosomal nucleic acids (exoNA) extracted from a biological fluid of a subject, a pair of amplification primers comprising a forward primer and a reverse primer, and a pair of detection primers comprising a mutant primer and a wild-type primer, wherein the mutant primer comprises a first detectable label and the wild-type primer comprises a second detectable label, under conditions sufficient for amplifying the TERT promoter sequence, and detecting a signal from the first detectable label and the second detectable label, wherein presence of the signal from the first detectable label indicates presence of a mutant TERT promoter sequence in the sample and/or wherein presence of the signal from the second detectable label indicates presence of a wild-type TERT promoter sequence in the sample, wherein the forward primer comprises SEQ ID NO: 1 and the reverse primer comprises SEQ ID NO: 2, wherein the mutant primer comprises SEQ ID NO: 3 and the wild-type primer comprises SEQ ID NO: 4, and wherein the mutant TERT promoter sequence comprises C228T or C250T.

In some embodiments, the DNA sample is extracted from the biological fluid using an ExoLution PLUS kit.

In some embodiments, the reaction mixture further comprises 7-deaza-2′-deoxyguanosine 5′-triphosphate (7-deaza-dGTP).

In some embodiments, the TERT promoter sequence is amplified by digital PCR (dPCR). In some embodiments, the TERT promoter sequence is amplified by droplet digital PCR (ddPCR).

In some embodiments, the mutant primer comprises at least one locked nucleic acid (LNA) modification and/or wherein the wild-type primer comprises at least one LNA modification. In some embodiments, the mutant primer comprises LNA modifications at positions 4, 5, 6, and 7 in SEQ ID NO: 3. In some embodiments, the wild-type primer comprises LNA modifications at positions 5, 6, and 7 in SEQ ID NO: 4.

In some embodiments, the forward primer is SEQ ID NO: 1. In some embodiments, the reverse primer is SEQ ID NO: 2. In some embodiments, the mutant primer is SEQ ID NO: 3. In some embodiments, the wild-type primer is SEQ ID NO: 4.

In some embodiments, the first detectable label comprises a first fluorophore and a first quencher. In some embodiments, the second detectable label comprises a second fluorophore and a second quencher. In some embodiments, the first fluorophore and the second fluorophore are different fluorophores. In some embodiments, the first quencher and the second quencher are the same quencher. In some embodiments, the first fluorophore and the second fluorophore are selected from the group consisting of FAM, HEX, Cy3, Cy5, and Texas Red. In some embodiments, the first quencher and the second quencher are selected from the group consisting of Iowa Black FQ, Iowa Black RQ, ZEN Quencher, and TAMRA.

In some embodiments, the subject is treatment naïve or wherein the subject has received a cancer therapy.

In some embodiments, the biological fluid is selected from the group consisting of plasma, urine, and cerebrospinal fluid (CSF).

In some embodiments, the subject is a human patient having or suspected of having a cancer. In some embodiments, the cancer is selected from brain cancer, skin cancer, lung cancer, liver cancer, breast cancer, thyroid cancer, adrenocortical carcinoma, ovarian cancer, endometrial carcinoma, renal cell carcinoma, bladder cancer, and gastric cancer. In some embodiments, the brain cancer is a glioma. In some embodiments, the glioma is selected from the group consisting of an astrocytoma, an ependymoma, and an oligodendroglioma.

In some embodiments, methods described herein further comprise administering a cancer therapy to the subject. In some embodiments, the cancer therapy is selected from the group consisting of a chemotherapy, a radiation therapy, a surgical therapy, and an immunotherapy.

Aspects of the present disclosure provide a method of evaluating reoccurrence of a cancer in a subject, the method comprising detecting the TERT promoter sequence in the DNA sample from the biological fluid of the subject according to any of the methods described herein, determining whether the subject has reoccurrence of the cancer, wherein the subject is identified as having reoccurrence of the cancer when the level of mutant TERT promoter sequences in the sample is higher than a control level, and administering a cancer therapy to the subject identified as having reoccurrence of the cancer.

Aspects of the present disclosure provide a method of evaluating effectiveness of a cancer therapy, the method comprising detecting the TERT promoter sequence in the DNA sample from the biological fluid of the subject according to any of the methods described herein, determining whether the cancer therapy has been effective, wherein the cancer therapy is identified as effective when the level of mutant TERT promoter sequences in the sample is higher than a control level, and administering the cancer therapy identified as effective to the subject and/or administering another cancer therapy to the subject.

Aspects of the present disclosure provide a pair of amplification primers for amplifying a promoter region of a telomerase reverse transcriptase (TERT) gene, the pair of amplification primers comprising a forward primer comprising SEQ ID NO: 1 and a reverse primer comprising SEQ ID NO: 2. In some embodiments, the present disclosure provides an amplification primer comprising SEQ ID NO: 2.

### BRIEF DESCRIPTION OF THE DRAWINGS

FIGS. 1A-1K include data from development of the TERT promoter mutation ddPCR assay. FIG. 1A includes a schematic depicting experimental workflow of studies described herein, including isolation of plasma, extraction platform, and TERT Promoter ddPCR readout. FIG. 1B includes a schematic of the TERT promoter nucleotide sequence illustrating forward and reverse primers as well as probes specific to each mutation. FIG. 1C includes a graph of absolute quantification of TERT mutant copies from equal inputs of genomic DNA from U87 (C228T), A431 (C250T), HBMVEC (WT) cell lines. FIG. 1D includes 2D amplitude plots indicating the mutant and wild-type populations for each specific mutation and cell line. Y-axis indicates positivity in the mutant channel, and the X-axis indicates positivity in the wild-type channel. Events positive for both channels are shown in the upper right corner of the 2D amplitude plot. Both mutant and wild-type probes can bind the C250T mutation, due to the location of the point mutation (positive in the lower and upper right corner). However, either the wild-type probe or the mutant probe can exclusively bind to the C228T mutation (positive in the upper left and lower right corner). Background signal is seen in the lower left corner. Serial dilutions of genomic DNA (gDNA) from A431 cells are used as templates for TERT ddPCR assay using 7-deaza-dGTP Q-sol as an additive. Copies per 20 μL of TERT Mutant (FIG. 1E) and TERT WT (FIG. 1F) plotted against input (in nanograms) of A431 gDNA. Serial dilutions of gDNA from TERT Mutant cell lines (FIG. 1G) U87 (C228T) and (FIG. 1H) A431 (C250T) were diluted in a constant background of TERT WT HBMVEC gDNA from 10% mutant allele frequency to 0% mutant allele frequency. Copies per 20 μL of TERT Mutant and TERT WT are plotted against mutant allele frequency. Limit of detection (LOD, dashed line) is plotted, defined as 2 standard deviations over the mean frequency abundance obtained at 0% when only TERT WT DNA was used as input. Limit of blank (LOB, dotted line) is plotted, defined as the highest apparent mean frequency abundance expected to be found when replicates of a blank sample containing no TERT Mutant copies are tested. FIG. 1I includes a graph showing copies of TERT WT/mL detected in healthy control plasma using two extraction platforms. cfDNA was extracted from 2 mL of healthy control plasma using the QIAmp circulating nucleic acid kit (QIAGEN) and the ExoLution PLUS extraction kit (Exosome Diagnostics). 2 μL of cfDNA was used as input for absolute quantification of TERT WT cfDNA. Copies/mL were calculated as described herein. FIGS. 1J-1K include data from absolute quantification of TERT WT from cfDNA extracted from 1 mL, 2 mL, and 4 mL of healthy control plasma using the ExoLution PLUS kit (Exosome Diagnostics). 2 μL of cfDNA was used as input for absolute quantification of TERT WT cfDNA. Copies per 20 μL (FIG. 1J) and Copies/mL (FIG. 1K) were plotted against the amount of healthy control plasma used for the reaction.

FIGS. 2A-2E include data from detection of TERT promoter mutation in plasma of Discovery Cohort. FIG. 2A includes a CONSORT diagram depicting patient cohorts and overall study design. FIG. 2B includes a graph of absolute quantification of TERT mutant (C228T or C250T) and wild-type copies in plasma samples from sample set 1. cfDNA from 2 mL of matched patient plasma from patient cohort 1 (21 TERT Mutant and 4 TERT WT; n=25) and healthy control (TERT WT; n=10) was used as input for absolute quantification of TERT mutant (C228T or C250T) and wild-type copies. FIG. 2C includes a graph of absolute quantification of TERT mutant (C228T or C250T) and wild-type copies in plasma samples from sample set 2. Plasma samples from PCR blinded sample set 2 (25 TERT Mutant and 13 TERT WT; n=38) and healthy control (TERT WT; n=10) were used as input for absolute quantification of TERT mutant (C228T or C250T) and analyzed using parameters established in sample set 1. All data is shown in MAF, calculated using the formula described in Methods and FIGS. 6A-6B. MAF of TERT Mutant for plasma samples are plotted against cohort sub-classification. Dotted line indicates a threshold of 0.26% MAF, used to designate samples as TERT Mutant positive or negative. FIG. 2D includes a graph of mutant allele frequency (MAF) of TERT Mutant according to sample number. Oncoprint depicting the genomic landscape of each sample are plotted underneath. FIG. 2E includes contingency tables, which were constructed from data obtained as described herein, and sensitivity and specificity calculated as described herein, and graphed above. Overall sensitivity and specificity across both sample sets (n=83) are also reported.

FIGS. 3A-3E include data from detection of TERT promoter mutation in plasma of blinded multi-institution validation cohort. FIG. 3A includes a CONSORT diagram depicting patient cohort and design of multi-institution validation cohort. FIG. 3B includes a graph of mutant allele frequency in plasma samples from patients in blinded multi-institution cohort. cfDNA from 2 mL of matched patient plasma from blinded multi-institution cohort (n=74; n=14 outside institution, TERT Mutant, n=28 MGH TERT Mutant, n=9 MGH TERT WT, n=14 MGH healthy control, n=9 Mill positive non-tumor) was used as input for absolute quantification of TERT mutant and analyzed using the parameters established in the discovery cohort. Data is shown in MAF calculated using the formula described in Methods and FIGS. 6A-6B. MAF for all samples is plotted against sub-classification of patient samples. FIG. 3C includes a graph of MAF for all TERT Mutant plasma samples graphed according to sample number, with an accompanying Oncoprint that depicts sample source and genomic landscape. Dotted line indicates threshold of 0.26% MAF used to designate samples as TERT Mutant positive or negative. FIG. 3D includes a graph of analytical parameters calculated from contingency tables. FIG. 3E includes a ROC Curve depicting change in sensitivity and specificity according to varying threshold. Black point indicates threshold used for analysis, 0.26% MAF. Gold Standard (MGH Pathology/SNapShot) is plotted in black, and TERT ddPCR Assay is plotted in gray.

FIGS. 4A-4E includes data from longitudinal monitoring of TERT promoter mutation in patients with glioma. TERT promoter mutation (copies/mL and MAF) in serial plasma samples obtained from five glioma patients are plotted against time (weeks-post-OP). Cases with stable disease include (FIG. 4A) P4 (MGH-19038; grade IV IDH1 wildtype GBM), and (FIG. 4B) P5 (MGH-19006; grade IV IDH1 wild-type GBM). Cases with progression include (FIG. 4C) P1 (MGH-18040; grade IV IDH1 wildtype GBM), (FIG. 4D) P3 (MGH-17045, grade IV IDH1 mutant GBM) and (FIG. 4E) P2 (MGH-18061; grade III, anaplastic astrocytoma). T1-Weighted, Contrast Enhanced MRI images are provided for timepoints when available. For P2, Axial Flair images are also provided. Timepoints are indicated as baseline (B), timepoint 1 (T1), timepoint 2 (T2), timepoint 3 (T3), and timepoint (T4). Surgical procedures are indicated using a square (STR=subtotal resection; GTR=gross total resection), a line indicates administration of chemoradiation and progression is indicated using a gray background.

FIGS. 5A-5B include data from detection of TERT promoter sequences. FIG. 5A includes a schematic depiction of detection of mutant TERT promoter sequences and wild-type promoter sequences. FIG. 5B includes a 2D amplitude plots for mutant allele frequencies, merging each replicate, for C250T (left) and C228T (right).

FIGS. 6A-6B include schematics showing R-based gating setting and detection of TERT promoter mutation in plasma of discovery, validation, and multi-institution cohorts. FIG. 6A includes a schematic depicting gating strategy and threshold determination using the training set (discovery and validation cohorts; n=83). FIG. 6B includes a schematic depicting blinded testing of validation set (multi-institution cohort; n=74) using gate and thresholds trained on discovery set.

FIGS. 7A-7D include data from tumor tissue analysis and analysis of plasma TERT in Copies/mL. FIG. 7A includes a graph showing absolute quantification of copies of TERT mutant and TERT WT from gDNA extracted from 21 TERT mutant and 4 TERT WT tumor tissue samples. 100 ng of tumor tissue gDNA was used as input for absolute quantification of copies of TERT mutant and TERT WT. Copies per 20 μL of TERT mutant and WT are plotted against Study ID, classified by SNapSHOT/Pathology. FIG. 7B includes data from 4 replicates of 4 L of cfDNA from matched plasma samples (21 TERT mutant and WT) and healthy control (10) was used as input for absolute quantification of TERT mutant and wild-type copies. Copies/mL were calculated using the formula described herein. Copies/mL for plasma samples are plotted against Study ID, classified by SNapSHOT/Pathology as floating bars, with line at the mean copies/mL. FIG. 7C includes data from samples group as to depict concordance between tumor tissue and matched plasma. FIG. 7D includes data of mean copies/mL of TERT mutant for plasma samples plotted against SNapSHOT/Pathology classification. Dotted line indicates threshold of 8.5 copies/mL, used to designate samples as mutant positive or negative.

FIGS. 8A-8L include correlation data graphs and contingency tables. Correlations are shown between TERT MAF and progression free survival (FIG. 8A), overall survival (FIG. 8B), tumor grade (FIG. 8C), contrast enhancement (FIG. 8D), type of TERT mutation (FIG. 8E), tumor volume (FIG. 8F), duration of disease (FIG. 8G), and age (FIG. 8H). Contingency tables are provided for Discovery Sample Set 1 (FIG. 8I), Discovery Sample Set 2 (FIG. 8J), Multi-Institution Validation Cohort (FIG. 8K), and Overall Combined Cohort (FIG. 8L).

FIGS. 9A-9B include data from detection of a TERT mutation in the CSF of a patient whose plasma TERT MAF was below the defined assay threshold. FIG. 9A includes a graph showing MAF (%). Four replicates of 4 μL of cfDNA from matched CSF samples (n=4; n=3 TERT mutant and n=1 WT) was used as input for absolute quantification of TERT mutant and wild-type copies. MAF is calculated using the formula described hereinin. Copies/mL for plasma samples are plotted against Study ID, classified by SNapSHOT/Pathology as floating bars, with line at the mean copies/mL. FIG. 9B includes a contingency table for matched CSF plasma.

FIGS. 10A-10B include data showing detection of mutant TERT promoter sequences in urine (FIG. 10A), saliva (FIG. 10A), and cerebrospinal fluid (CSF) (FIG. 10B).

The details of one or more embodiments of the invention are set forth in the description below. Other features or advantages of the present invention will be apparent from the following drawings and detailed description of several embodiments, and also from the appended claims.

### DETAILED DESCRIPTION

The present disclosure is based, at least in part, on the development of improved methods for detecting a mutant TERT promoter sequence, particularly C228T and C250T mutations, including improved conditions for one or more steps of the detection method. The improved detection methods disclosed herein led to at least the following advantageous outcomes:

(a) Improved sensitivity resulting at least in part from enhanced TERT sequence recovery provided by the improved nucleic acid extraction conditions provided herein.

(b) Improved specificity resulting at least in part from four-fold higher absolute mutant detection and comparable wild-type detection provided by the improved amplification conditions provided herein.

(c) Improved concordance between liquid biopsy samples and tissue biopsy samples resulting from the improved assay conditions provided herein.

(d) Low rates of false positives resulting from the improved assay conditions provided herein.

(e) High sensitivity and high specificity that allows longitudinal monitoring during the course of therapy, thereby eliminating the need for multiple invasive biopsies, which can be challenging for brain cancer patients.

Accordingly, provided herein are methods for detecting mutant TERT promoter sequences (e.g., C228T, C250T) in biological samples from a subject. Methods provided herein can also be useful for evaluating reoccurrence of a cancer and/or evaluating efficacy of a cancer therapy. Compositions comprising a pair of amplification primers are also within the scope of the present disclosure.

TERT promoter mutations are highly prevalent in gliomas (>60%), with the highest incidence in primary glioblastomas (>80%). Detection and quantification of TERT promoter mutations in cell-free DNA (cfDNA) has been previously investigated for several cancers with limited success due, at least in part, to low sensitivity and specificity of the assays and low abundance of cfDNA in the sample.

Methods described herein overcome several challenges that have prevented detection of cfDNA point mutations in specific genes in the plasma of brain tumor patients, including mutations in the TERT promoter. In contrast to other tumor types, where cfDNA point mutations are abundant, such is not the case in glioma. Several prior reports have detected specific oncogenic point mutations in cfDNA, including at the TERT promoter locus, in less than 5% of patients. This may be related to generally lower levels of CNS-derived circulating tumor DNA (ctDNA) in the blood, thought to be a function of the blood brain barrier.

Methods described herein demonstrate that tumor size does not correlate with plasma TERT mutant allele frequency (MAF). However, it was observed that patients with contrast enhancing lesions (indicative of BBB breakdown) were more likely to have higher plasma TERT MAF. Given that recurrence timepoints (after surgery and chemoradiation) have more edema and higher plasma TERT MAF compared to baseline despite smaller tumor volumes, it is possible that plasma TERT MAF is correlated with breakdown of the blood brain barrier.

Methods described herein included several technical developments to detect low concentrations of TERT promoter mutations in plasma. Both C228T and C250T generate identical sequences, which can be detected with a single probe with LNA enhancements that stabilize probe-template duplexes to improve SNV discrimination. Furthermore, the TERT promoter region has a high GC-content (>80%) which was addressed by using 7-deaza-dGTP as a ddPCR additive, which interrupts the formation of these secondary structures. In combination, with standardized handling strategies and an unbiased analytic method, methods described herein provide a significant improvement in assay sensitivity over previously published reports for TERT promoter detection in plasma of patients with glioma.

The challenge in detecting plasma cfDNA from brain tumor patients highlights a missed opportunity for the advantages of the liquid biopsy approach in a tumor type where initial or repeat direct tissue sampling may pose substantial neurologic risk. The ability to detect oncogenic point mutations in the blood can have at least two near-term applications. For example, methods described herein can be useful for upfront diagnosis. Because some malignant tumors are located in deep or inaccessible locations and would be poor surgical candidates for direct tissue biopsy or resection, the combination of a characteristic set of MRI imaging findings in addition to a liquid biopsy that shows TERT promoter mutation can be sufficient to establish a high positive predictive value for the clinical diagnosis malignant glioma and allow adjunctive treatment with radiation and chemotherapy to proceed with confidence.

In another example, because of the surgical risk of repeated brain biopsies, a liquid biopsy approach to TERT promoter mutation detection in the blood may be particularly suitable for therapeutic monitoring of disease burden, as exemplified in the longitudinal studies described herein. As described herein, levels of TERT promoter mutants, detected as described herein, correlate with disease recurrence or progression. Such information can aid in differentiation between radiation necrosis, pseudoprogression, and true progression, thus minimizing the need for further invasive workup and improving overall quality of care. Furthermore, with emerging insights into intratumoral heterogeneity, the validity of a single, localized tissue biopsy as a true gold standard has begun to be debated, raising the possibility that a liquid biopsy, which effectively samples multiple tumor regions as blood perfuses the solid tumor mass, may offer greater sensitivity for TERT promoter mutation than a single, focal, tissue biopsy, in some patients.

Methods described herein, in some embodiments, provide a novel and highly sensitive ddPCR based TERT promoter mutation assay that utilizes high affinity LNA enhanced probes and the additive 7dG to reduce the formation of secondary structures. Such improvements enable detection and monitoring of TERT promoter mutations (e.g., C228T, C250T) in tumor tissue and cfDNA of matched plasma of glioma patients with an overall sensitivity of 62.5% and a specificity of 90% in combined discovery and blinded validation cohorts of 157 samples. The ability to detect TERT mutations, which are highly prevalent in glioma patients, in the plasma enhances the ability to diagnose, monitor and assess response to therapy. Liquid biopsy-based monitoring can significantly impact clinical care by guiding patient stratification for clinical trials, offering new opportunities for the development of targeted therapies ultimately improving patient care.

### I. Components for Detection of TERT Promoter Mutations

Methods described herein involve use of a pair of amplification primers and a pair of detection primers to detect TERT promoter mutations with high sensitivity and high specificity in a sample (e.g., a plasma sample).

(a) Amplification Primers

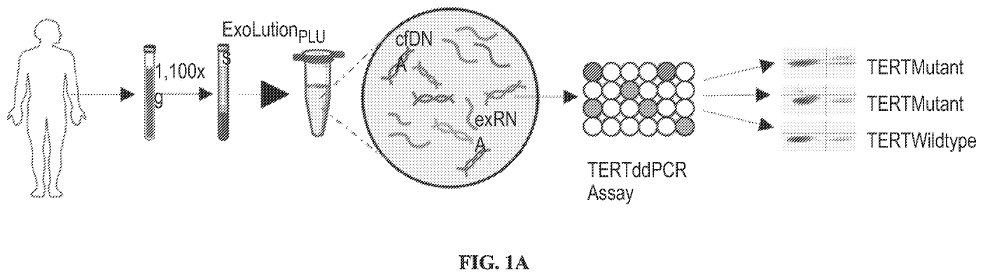

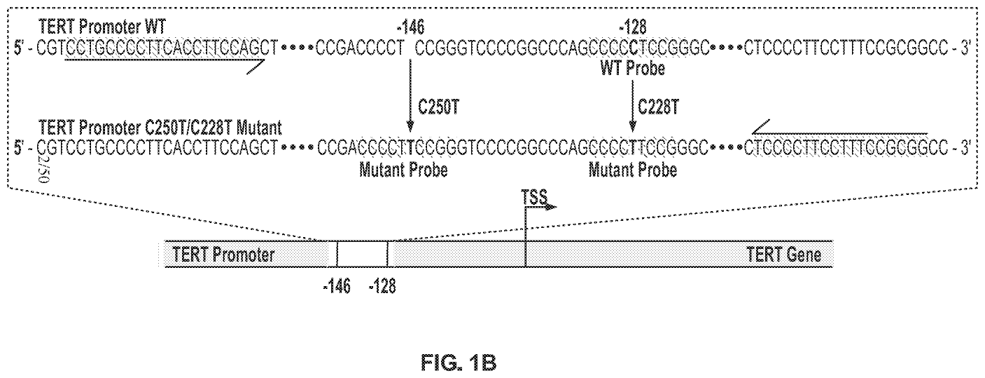

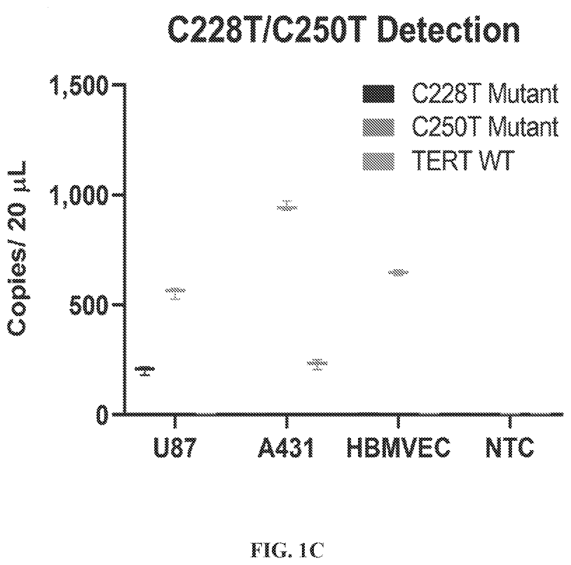

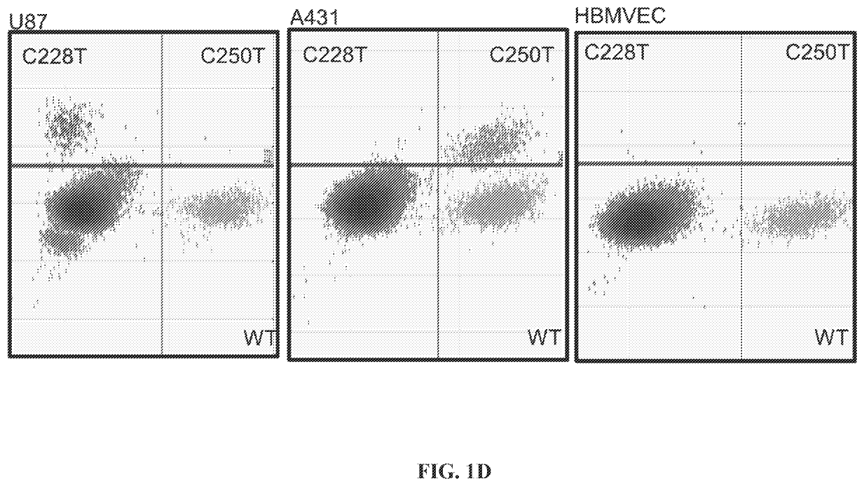

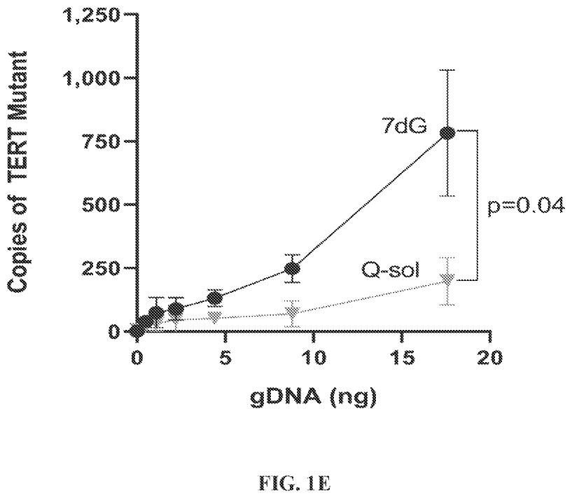

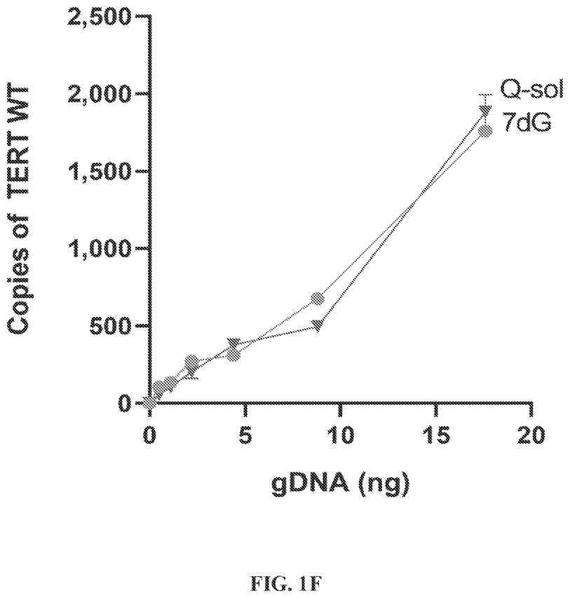

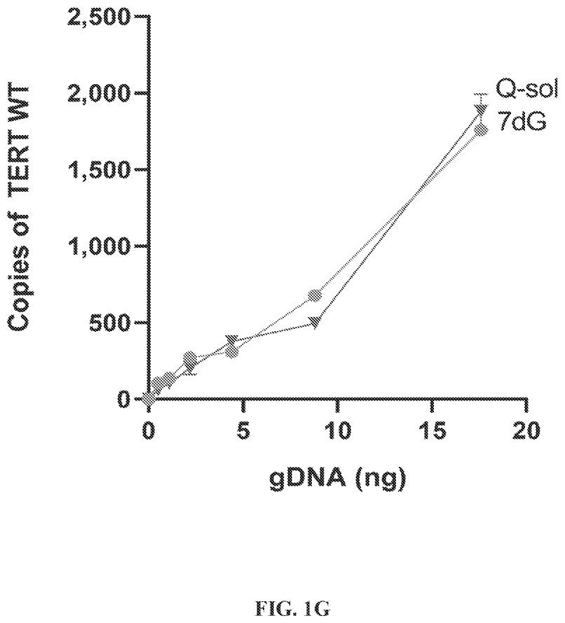

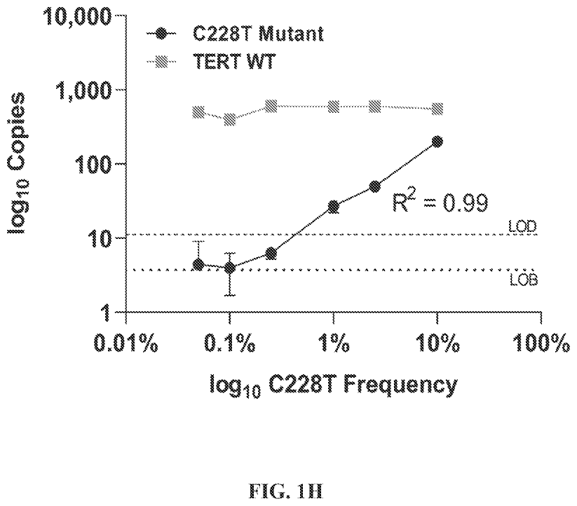

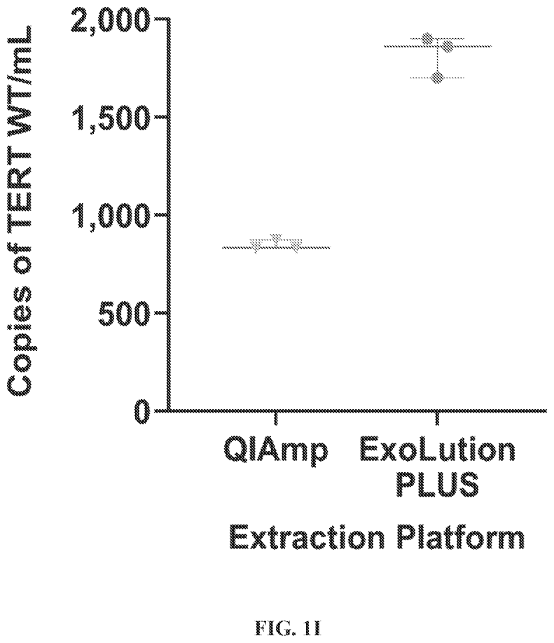

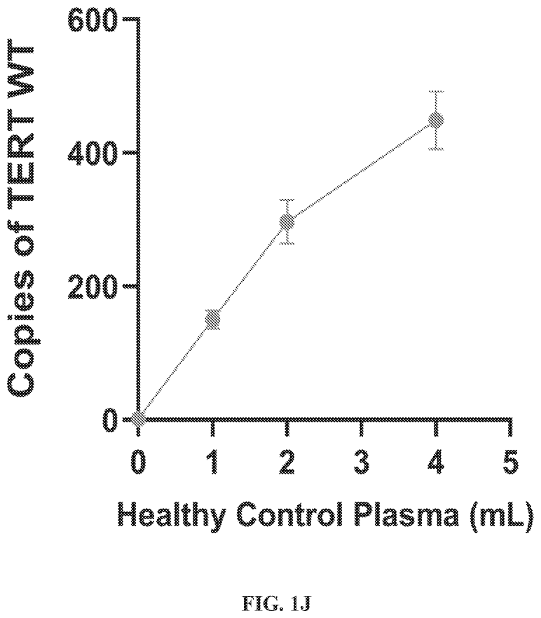

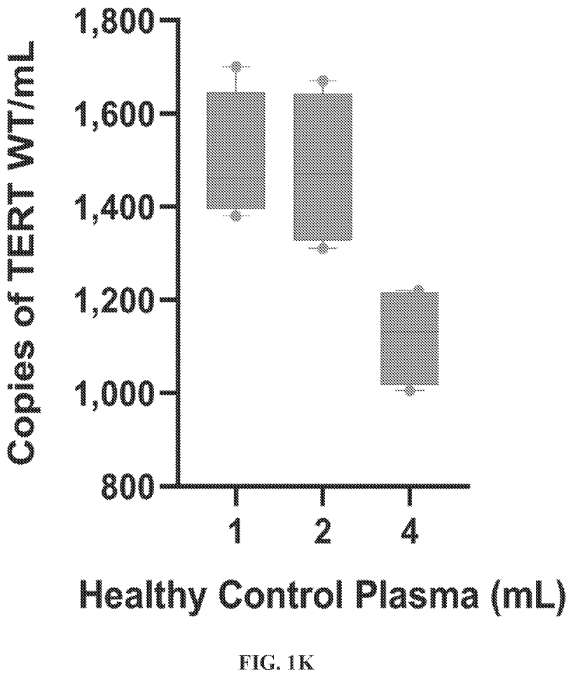

Methods described herein involve amplification of a promoter region of a telomerase reverse transcriptase (TERT) gene using a pair of amplification primers, which comprises a forward primer and a reverse primer. An example of a pair of amplification primers comprising a forward primer (e.g., SEQ ID NO: 1) and a reverse primer (e.g., SEQ ID NO: 2) flanking (i.e., one on either side) a portion of a TERT promoter sequence is shown in FIG. 1B.

In other examples, the forward primer comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 1. In some embodiments, the forward primer comprises SEQ ID NO: 1. In some embodiments, the forward primer is SEQ ID NO: 1.

In other examples, the reverse primer comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 2. In some embodiments, the reverse primer comprises SEQ ID NO: 2. In some embodiments, the reverse primer is SEQ ID NO: 2.

It should be understood that the forward primer and/or the reverse primer can comprise a modified nucleotide such as those known in the art or described herein. Modified nucleotides include, but are not limited to, nucleotides comprising a backbone modification, a base modification, and/or a sugar modification. Non-limiting examples of backbone modifications include phosphorothioate modifications, methylphosphonate modification, phosphoramidate modifications, and locked nucleic acid (LNA) backbone modifications. Non-limiting examples of base modifications include substituted purines and pyrimidines. Non-limiting examples of sugar modifications include 2′-O-alkylated or 2′-fluorinated ribose and arabinose. Other such modifications are well known to those of skill in the art.

In some embodiments, the mutant primer comprises at least 1, at least 2, at least 3, at least 4, at least 5, at least 6, at least 7, at least 8, at least 9, at least 10, or more modified nucleotides. In some embodiments, the wild-type primer comprises at least 1, at least 2, at least 3, at least 4, at least 5, at least 6, at least 7, at least 8, at least 9, at least 10, or more modified nucleotides.

(b) Detection Primers

Methods described herein involve detection of wild-type and mutant (e.g., C228T or C250T) sequences in a promoter region of a telomerase reverse transcriptase (TERT) gene using a pair of detection primers, which comprises a mutant primer and a wild-type primer. An example of a pair of detection primers comprising a mutant primer (e.g., SEQ ID NO: 3) and a wild-type primer (e.g., SEQ ID NO: 4) is shown in FIG. 1B.

The mutant primer described herein can simultaneously detect the most common TERT promoter mutations, C228T and C250T. In some embodiments, the mutant primer comprises SEQ ID NO: 3. In some embodiments, the mutant primer is SEQ ID NO: 3.

The wild-type primer described herein can detect a wild-type TERT promoter sequence (e.g., C228). In some embodiments, the wild-type primer comprises SEQ ID NO: 4. In some embodiments, the wild-type primer is SEQ ID NO: 4.

In some embodiments, stabilization of primer-template duplexes (e.g., mutant primer-template, wild-type primer-template) can be achieved using a nucleotide modification such as a locked nucleic acid (LNA) modification. In some embodiments, the detection primers disclosed herein comprise at least 1, at least 2, at least 3, at least 4, at least 5, or more nucleotides comprising a locked nucleic acid (LNA) modification. For example, the mutant primer comprises SEQ ID NO: 3 comprising a LNA modification at position 4, 5, 6, and 7. In another example, the wild-type primer comprises SEQ ID NO: 4 comprising a LNA modification at position 5, 6, and 7.

Detection primers disclosed herein can comprise a detectable label for quantification of wild-type and mutant TERT promoter sequences. As used here, a detectable label refers to any molecule that is capable of releasing a detectable signal, either directly or indirectly. Any detectable label known in the art can be incorporated into a detection primer described herein.

Examples of detectable labels include, but are not limited to, fluorescent dyes (e.g., fluorophores), affinity tags (e.g., biotin), luminescent agents, electron-dense reagents, enzymes (e.g., luciferase), isotopes (e.g., ³²P), haptens, and proteins. The detection primers can be labeled using any method known in the art (e.g., click chemistry).

A fluorophore, as used herein, refers to a molecule with a fluorescent emission maximum between about 350 and about 900 nm. Any suitable fluorophore may be used to label detection primers described herein. Examples of fluorophores include, but are not limited to, 5-FAM (5-carboxyfluorescein), HEX (Hexachloro-fluorescein), Cy5 (Indodicarbocyanine-5), Cy3 (Indo-dicarbocyanine-3), and Texas Red (Sulforhodamine 101 acid chloride).

A quencher, as used herein, refers to a molecule or part of a compound that is capable of reducing the signal (e.g., fluorescence) of a detectable label (e.g., a fluorophore) when attached to or in proximity to the detectable label. Quenching can occur by any mechanism including, but not limited to, fluorescence resonance energy transfer and photo-induced electron transfer. Fluorescence can be “quenched when the fluorescence emitted by the fluorophore is reduced as compared with the fluorescence in the absence of the quencher by at least 10%, e.g., 15%, 20%, 30%, 40%, 50%, 60%, 70%, 80%, 90%, 95%, 98%, 99%, 99.9% or more. The selection of the quencher can depend on the fluorophore used. A number of commercially available quenchers are known in the art, and include but are not limited to DABCYL, Black Hole™ Quenchers (e.g., BHQ-1, BHQ-2, and BHQ-3), Iowa Black™ FQ, and Iowa Black™ RQ.

In some embodiments, the detectable label comprises a fluorophore and a quencher pair. For example, the mutant primer comprises a first fluorophore and a first quencher. In another example, the wild-type primer comprises a second fluorophore and a second quencher. In such instances, the first fluorophore and the second fluorophore are different, thereby providing distinguishable signals. The first quencher and the second quencher may be the same or different.

The detectable label (e.g., a fluorophore and quencher pair) can be attached to the detection primer using any method known in the art. The detectable label can be attached to any portion of the detection primer. For example, when the detectable label is a fluorophore and quencher pair, one of the fluorophore and quencher pair is attached to the 5′ portion of the detection primer and the other of the fluorophore and quencher pair is attached to the 3′ portion of the detection primer.

(c) Biological Samples

Methods described herein involve detecting a TERT promoter sequence in a biological sample, e.g., a biological sample from a subject. Any sample that may contain a TERT promoter sequence can be analyzed by methods described herein. In particular, samples comprising low levels (e.g., ng quantities or less) of a TERT promoter sequence (e.g., plasma samples) can be analyzed by methods described herein. See Examples below.

Methods described herein can include providing a sample obtained from a subject. In some examples, the sample may be from an in vitro assay, for example, and in vitro cell culture (e.g., A431, U87, HBMVEC cells). As used herein, a sample refers to a composition comprising biological materials including, but not limited to, tissue, cells, and/or fluid from a subject. A sample includes both an initial unprocessed sample taken from a subject as well as subsequently processed, e.g., partially purified or preserved forms. In some embodiments, the sample is tissue such as tumor tissue. In other examples, the sample is a body fluid such as plasma, urine, and/or cerebrospinal fluid (CSF). In some embodiments, multiple (e.g., at least 2, 3, 4, 5, or more) samples may be collected from a subject, over time or at particular time intervals, for example, to assess the disease progression or evaluate the efficacy of a treatment.

A sample can be obtained from a subject using any means known in the art. In some embodiments, the sample is obtained from the subject by a surgical procedure (e.g., brain surgery). In some embodiments, the sample is obtained from the subject by a biopsy (e.g., a stereotactic needle biopsy). In some embodiments, the sample is obtained from a human.

A sample can be processed using nucleic acid extraction conditions provided herein to achieve higher concentrations of TERT promoter sequences from the same amount of starting material than can be achieved using other conditions. Such nucleic acid extraction conditions involve isolation of cell-free DNA (cfDNA) and exosomal nucleic acids (exoNA) from the biological sample. In other examples, genomic DNA (gDNA) is extracted from the biological sample using any suitable method known in the art or described herein.

In some embodiments, nucleic acid extraction conditions comprise contacting the biological sample with an anion exchange membrane or anion exchange bead and polyethylene glycol, contacting the membrane or the bead with a guanidine thiocyanate-based elution buffer, which releases the nucleic acids from the membrane or beads to produce a homogenate, and contacting the homogenate with a silica-based solid surface, thereby extracting the nucleic acids from the homogenate. In some embodiments, nucleic acid extraction conditions further comprise adding a protein precipitation buffer to the homogenate prior to extraction of the nucleic acid from the homogenate.

A non-limiting example of nucleic acid extraction conditions that can be used as provide herein are provided in U.S. Pat. No. 10,808,240, the relevant disclosures of which are herein incorporated by reference for the purposes and subject matter referenced herein. Alternatively, or in addition to, nucleic acid extraction conditions provided herein can be achieved using a commercially available kit such as ExoLution PLUS (Exosome Diagnostics).

### II. Methods for Detection of TERT Promoter Mutations

To perform the assay methods disclosed herein, a sample suspected of containing a mutant TERT promoter sequence can be brought in contact with the pair of amplification primers and the pair of detection primers under conditions suitable for amplification of the TERT promoter sequence. In such instances, the sample, the pair of amplification primers, and the pair of detection primers can be contacted in a reaction mixture.

As used herein, the term “contacts” refers to an exposure of a biological sample with a pair of amplification primers and a pair of detection primers under conditions suitable for amplification of a TERT promoter sequence (e.g., mutant TERT sequence and/or wild-type TERT sequence), if any. Amplification can be performed using PCR (e.g., digital PCR (dPCR), droplet digital PCR (ddPCR)).

A reaction mixture can be incubated under conditions sufficient for amplification of a TERT promoter sequence. The amplification step used in any of the methods disclosed herein can involve amplification conditions disclosed herein that provide high sensitivity and high specificity of detection. See Examples below.

In some embodiments, amplification of a TERT promoter sequence can be achieved using a stabilizing agent such as 7-deaza-2′-deoxyguanosine 5′-triphosphate (7-deaza-dGTP; 7dG). In some embodiments, the reaction mixture comprises 50 to 500 mM 7dG. In some embodiments, the reaction mixture comprises 100 to 500 mM, 200 to 500 mM, 300 to 500 mM, 400 to 500 mM, 50 to 400 mM, 50 to 300 mM, 50 to 200 mM, or 50 to 100 mM 7dG.

Presence or level (e.g., amount such as copies/mL) of mutant TERT promoter sequence in the sample can be detected by measuring a signal released from the detectable label attached to the mutant primer. Alternatively, or in addition to, presence or level of wild-type TERT promoter sequence in the sample can be detected by measuring a signal released from the detectable label attached to the wild-type primer. The detectable labels attached to the mutant primer and the wild-type primer can be different, thereby providing distinguishable signals.

As used herein, the terms “detecting” or “determining” or “measuring” can include assessing the presence, absence, quantity and/or amount of a TERT promoter sequence in a sample, including the derivation of qualitative or quantitative concentration levels of the TERT promoter sequence, or otherwise evaluating the values and/or categorizing the values of the TERT promoter sequence in a sample from the subject.

Methods described herein, in some embodiments, encompass an extraction step in which nucleic acids (e.g., cfDNA and exoNA) are extracted from a biological sample using nucleic acid extraction conditions provided herein to achieve higher concentrations of TERT promoter sequences from the same amount of starting material than can be achieved using other conditions.

Accordingly, in some embodiments, methods described herein comprise extracting nucleic acids (e.g., cfDNA and exoNA) from a biological sample from a subject to obtain a DNA sample comprising a TERT promoter sequence, incubating the DNA sample with a pair of amplification primers, and a pair of detection primers under conditions sufficient for amplifying the TERT promoter sequence, and detecting a signal from each of the detection primers.

In some embodiments, extracting nucleic acids from a biological sample from a subject comprises contacting the biological sample with an anion exchange membrane or anion exchange bead and polyethylene glycol, contacting the membrane or the bead with a guanidine thiocyanate-based elution buffer, which releases the nucleic acids from the membrane or beads to produce a homogenate, and contacting the homogenate with a silica-based solid surface, thereby extracting the nucleic acids from the homogenate. In some embodiments, extracting nucleic acids from a biological sample from a subject further comprises adding a protein precipitation buffer to the homogenate prior to extraction of the nucleic acid from the homogenate. In some embodiments, extracting nucleic acids from a biological sample from a subject comprises extracting nucleic acids using a commercially available kit such as ExoLution PLUS (Exosome Diagnostics).

Assays can be performed on low-throughput platforms, including single assay format. Alternatively, or in addition to, assays may be performed on high-throughput platforms. The type of platform used for the detection and/or quantification of a TERT promoter sequence may depend on the particular situation in which the assay is to be used (e.g., clinical or research applications), on the kind and number of patient samples to be run in parallel, to name a few parameters.

The assay methods described herein can be used for both clinical and non-clinical purposes. Some examples are provided herein.

### III. Application of Methods for Detection of TERT Promoter Mutations

Methods described herein can be applied for evaluation of cancer, e.g., diagnosis or prognosis of a cancer. Evaluation can include identifying a subject as being at risk for or having a cancer as described herein, e.g., glioma. Evaluation can also include monitoring treatment of a cancer, such as evaluating the effectiveness of a treatment for a cancer, and/or monitoring reoccurrence of a cancer. Methods described herein can also be applied for non-clinical applications such as for research purposes.

(a) Diagnosis and Prognosis

Methods described herein are used to determine the level of mutant TERT promoter sequence (e.g., C228T or C250T) in a sample (e.g., a serum sample or a plasma sample or a blood sample) collected from a subject (e.g., a human patient suspected of having a cancer such as glioma). For example, the level of mutant TERT promoter sequence can be quantified as TERT mutant copies/mL of plasma. The level of mutant TERT promoter sequence can then be compared to a reference value to determine whether the subject has or is at risk for a cancer. The reference value can be a control level of mutant TERT promoter sequence or a level of wild-type TERT promoter sequence. In some embodiments, the control level is a level of mutant TERT promoter sequence or wild-type TERT promoter sequence in a control sample (e.g., a sample obtained from a healthy subject or population of healthy subjects). As used herein, a healthy subject refers to a subject that is apparently free of a cancer at the time the level of TERT is measured or has no history of a cancer.

The control level can also be a predetermined level. Such a predetermined level can represent a level of mutant TERT promoter sequence in a population of subjects that do not have or are not at risk for a cancer. The predetermined level can take a variety of forms. For example, it can be a single cut-off value, such as a median or mean. In some embodiments, such a predetermined level can be established based upon comparative groups, such as where one defined group is known to have a cancer and another defined group is known not to have a cancer (e.g., a healthy individual). Alternatively, or in addition to, the predetermined level can be a range including, for example, a range representing the levels of mutant TERT promoter sequence and/or wild-type TERT promoter sequence in a control population.

The control level as described herein can be determined as described herein and/or by a technology known in the art. In some examples, the control level can be obtained by performing a known method on a control sample as also described herein. In some embodiments, the control level can be obtained from members of a control population (e.g., healthy individuals) and the results can be analyzed by, for example, a computer program, to obtain the control level (a predetermined level) that represents the level of wild-type TERT promoter sequence and/or mutant TERT promoter sequence in the control population.

By comparing the level of wild-type TERT promoter sequence and mutant TERT promoter sequence in a sample obtained from a subject to a reference value as described herein, it can be determined as to whether the subject has or is at risk for a cancer (e.g., glioma). For example, if the level of mutant TERT promoter sequence in a sample obtained from a candidate subject increased as compared to the reference value (e.g., the level of mutant TERT promoter sequence in a sample from a healthy control), the candidate subject might be identified as having or at risk for a cancer. Alternatively, or in addition to, if the level of wild-type TERT promoter sequence in a sample obtained from a candidate subject decreased as compared to the reference value (e.g., the level of wild-type TERT promoter sequence in a sample from a healthy control), the candidate subject might be identified as having or at risk for a cancer.

As used herein, “an elevated level” or “a level above a reference value” means that the level of mutant TERT promoter sequence is higher than a reference value, such as a predetermined threshold or a level of mutant TERT promoter sequence in a control sample. An elevated or increased level of mutant TERT promoter sequence includes a level of mutant TERT promoter sequence that is, for example, 1%, 5%, 10%, 20%, 30%, 40%, 50%, 60%, 70%, 80%, 90%, 100%, 150%, 200%, 300%, 400%, 500% or more above a reference value. An elevated level of the mutant TERT promoter sequence also includes increasing a level from a zero state (e.g., no or undetectable mutant TERT promoter sequence in a sample) to a non-zero state (e.g., some or detectable mutant TERT promoter sequence in the sample).

As used herein, “a decreased level” or “a level below a reference value” means that the level of wild-type TERT promoter sequences is lower than a reference value, such as a predetermined threshold or a level of the wild-type TERT promoter sequence in a control sample. An reduced or decreased level of the wild-type TERT promoter sequence includes a level of wild-type TERT promoter sequence that is, for example, 1%, 5%, 10%, 20%, 30%, 40%, 50%, 60%, 70%, 80%, 90%, 100%, 150%, 200%, 300%, 400%, 500% or more below a reference value. A decreased level of the wild-type TERT promoter sequence also includes decreasing a level from a non-zero state (e.g., some or detectable wild-type TERT promoter sequence in the sample) to a zero state (e.g., no or undetectable mutant TERT promoter sequence in a sample).

In some embodiments, the subject is a human patient having, suspected of having, or at risk for having a cancer. A subject might show one or more symptoms of a cancer, e.g., fatigue, a lump or area of thickening that can be felt under the skin, weight changes (e.g., unintended weight loss or weight gain), skin changes (e.g., yellowing, darkening or redness of the skin, sores that cannot heal, or changes to existing moles), changes in bowel or bladder habits, persistent cough or trouble breathing, persistent muscle or joint pain, persistent fevers or night sweats, and unexplained bleeding or bruising.

Alternatively, or in addition to, the subject might show one or more symptoms of a glioma, e.g., headache, memory loss, urinary incontinence, seizures, speech difficulties, confusion, and balance difficulties.

A sample may be obtained from a subject having, suspected of having, or at risk for having a cancer. In some embodiments, the subject has a symptom of a cancer (e.g., glioma) at the time the sample is collected, has no history of a symptom of a cancer, or no history of a cancer. In some embodiments, the subject is resistant to a cancer treatment.

(b) Evaluation of Cancer Reoccurrence and Treatment Effectiveness

Methods described herein can also be applied to evaluate the reoccurrence of a cancer and/or to evaluate effectiveness of a cancer therapy. For example, multiple samples (e.g., serum or plasma samples) can be collected from a subject to whom a treatment is performed either before and after the treatment or during the course of the treatment.

If the level of mutant TERT promoter sequences (e.g., C228T or C250T) increases after the treatment or over the course of the treatment (level of mutant TERT promoter sequences in a later collected sample as compared to that in an earlier collected sample), remains the same or increases, it indicates that the cancer has reoccurred and/or that the treatment is ineffective.

If the level of mutant TERT promoter sequences (e.g., C228T or C250T) decreases after the treatment or over the course of the treatment (level of mutant TERT promoter sequences in a later collected sample as compared to that in an earlier collected sample), remains the same or decreases, it indicates that the cancer has not reoccurred and/or that the treatment is effective.

Alternatively, or in addition to, if the level of wild-type TERT promoter sequences decreases after the treatment or over the course of the treatment (level of wild-type TERT promoter sequences in a later collected sample as compared to that in an earlier collected sample), remains the same or decreases, it indicates that the cancer has occurred and/or that the treatment is ineffective.

If the level of wild-type TERT promoter sequences increases after the treatment or over the course of the treatment (level of wild-type TERT promoter sequences in a later collected sample as compared to that in an earlier collected sample), remains the same or increases, it indicates that the cancer has not reoccurred and/or that the treatment is effective.

If the subject is identified as not responsive to the treatment and/or as having a reoccurrence of the cancer, a higher dose and/or frequency of dosage of the therapeutic agent can be administered to the subject. In some embodiments, the dosage and/or frequency of dosage of the therapy is maintained, lowered, or ceased in a subject identified as responsive to the treatment or not in need of further treatment. Alternatively, a different treatment can be applied to the subject who is found as not responsive to the first treatment and/or who is identified as having reoccurrence of the cancer.

(c) Non-Clinical Applications

Methods described herein can also be applied to non-clinical uses, e.g., for research purposes. For example, methods described herein can be used to study cancer cell behavior and/or cancer cell mechanisms, which can identify novel biological pathways or processes involved in cancer (e.g., cancer development and/or cancer metastasis).

In some embodiments, methods described herein can be applied to the development of new therapy. For example, the levels of TERT promoter mutations can be measured in samples obtained from a subject having been administered a new therapy (e.g., in a clinical trial). In some embodiments, the level of TERT promoter mutations can indicate the efficacy of the new therapy or the progress of the cancer in the subject prior to, during, or after the new therapy.

### IV. Treatment of a Cancer

A subject identified as having, suspected of having, or at risk for having a cancer (e.g., glioma), as identified using the methods described herein, can be treated with any appropriate therapy. In some embodiments, methods provided herein include administering a cancer therapy to a subject based on the output of the methods described herein, e.g., detecting a TERT promoter mutation. Examples of a cancer therapy include, but are not limited to, a chemotherapy, a radiation therapy, a surgical therapy, and an immunotherapy.

In some embodiments, a chemotherapy is administered to the subject. Chemotherapy includes, but is not limited to, alkylating agents (e.g., Cisplatin), nitrosoureas (e.g., Carmustine, Lomustine, Streptozocin), antimetabolites (e.g., Gemcitabine, Hydroxyurea, Methotrexate), anti-tumor antibiotics (e.g., anthracyclines such as Doxorubicin), Topoisomerase inhibitors (e.g., camptothecins, epipodophyllotoxins), mitotic inhibitors (e.g., taxanes, vinca alkaloids), and corticosteroids (e.g., Prednisone, Methylprednisolone, Dexamethasone)

In some embodiments, a radiation therapy is administered to the subject. Radiation therapy includes, but is not limited to, ionizing radiation, gamma-radiation, neutron beam radiotherapy, electron beam radiotherapy, proton therapy, brachytherapy, and radioactive isotopes and radiosensitizers.

In some embodiments, a surgical therapy is administered to the subject. Surgical therapy includes, but is not limited to, curative surgery (e.g., tumor removal surgery), a preventive surgery, a laparoscopic surgery, and laser surgery.

In some embodiments, an immunotherapy is administered to the subject. Immunotherapy includes, but is not limited to, adoptive cell therapy, cancer vaccine therapy, immune checkpoint inhibitors (e.g., PD-1 inhibitors or PD-L1 inhibitors), oncolytic virus therapy, targeted antibody therapy, and immune-modulating therapy (e.g., cytokine therapy).

Methods described herein comprise administering one type of cancer therapy or multiple types of cancer therapies, which can be referred to as combination therapy. For example, the subject can be treated using a chemotherapy and an immunotherapy. It should be appreciated that any combination of cancer therapies can be administered to the subject. A cancer therapy can be administered one or more times to the subject.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Examples

In order that the invention described may be more fully understood, the following examples are set forth. The examples described in this application are offered to illustrate the methods and compositions provided herein and are not to be construed in any way as limiting their scope.

### Materials and Methods

The following materials and methods were used in the Examples set forth herein.

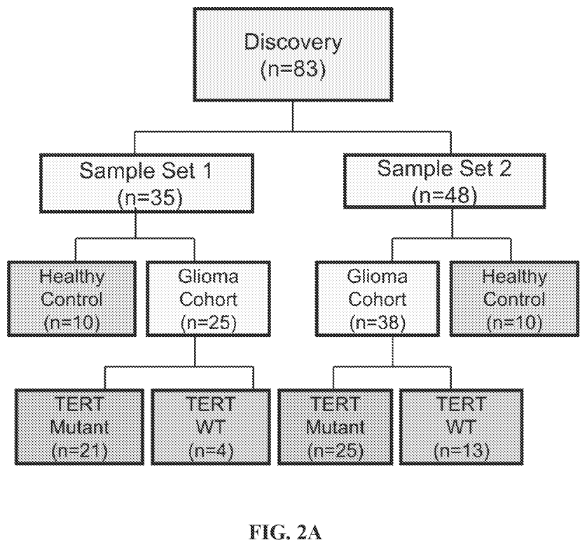

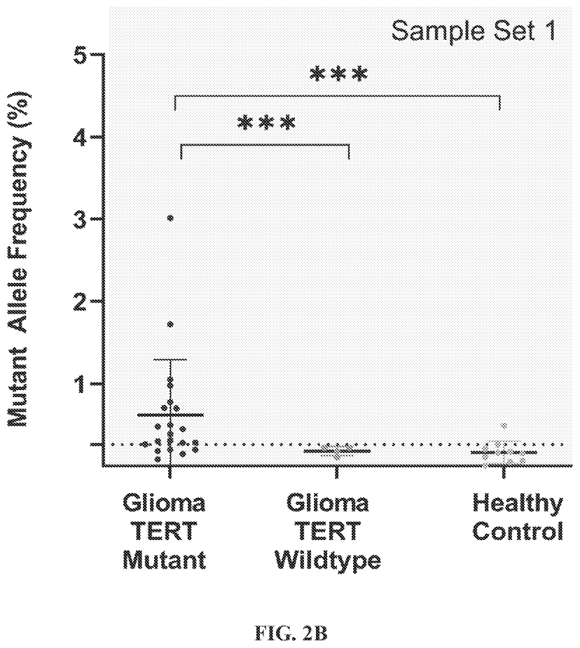

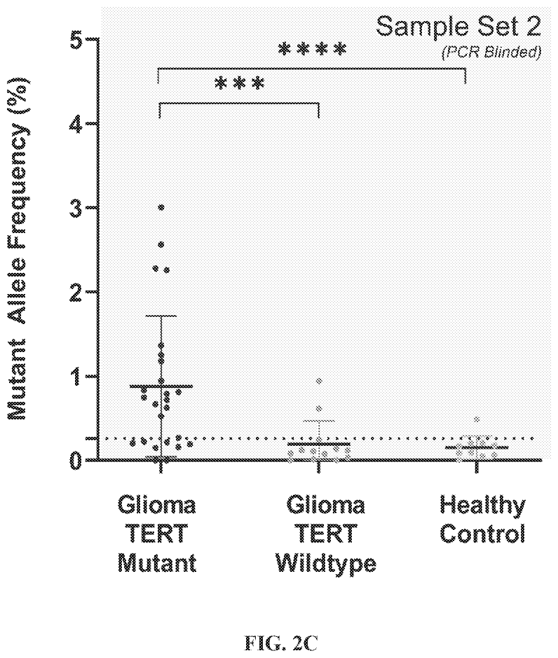

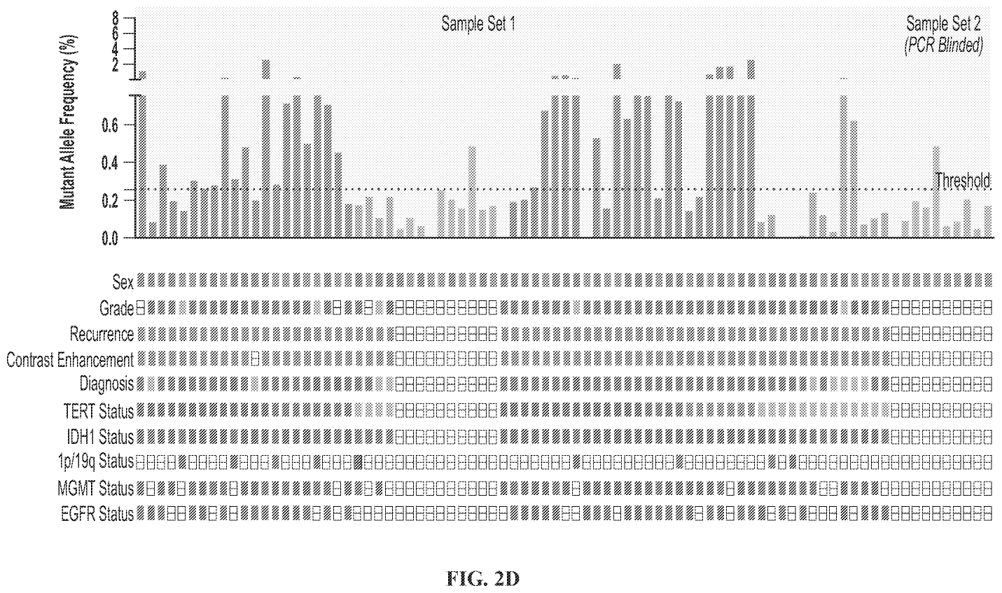

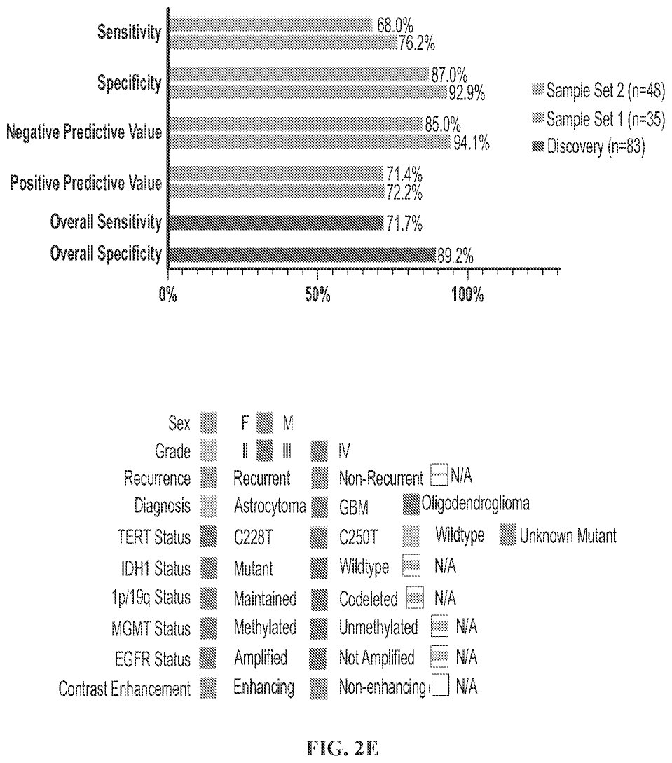

Study population. The study population (n=157) included patients 18 years or older with pathology confirmed TERT mutant or wildtype gliomas who underwent surgery at either the Massachusetts General Hospital (MGH), Henry Ford Healthy System (HFHS), or at Washington University St. Louis (WUStL) and age-matched healthy controls (FIG. 2A; Table 1). The study population was divided into a discovery cohort (n=83; 46 TERT Mutant, 17 TERT wild-type, 20 matched healthy controls) and a blinded multi-institution validation cohort (n=74; 42 TERT Mutant, 9 TERT WT, 14 matched healthy controls, 9 non-tumor Mill positive). For glioma cohorts, exclusion criteria consisted of history of other primary or metastatic cancers, active infectious disease, current or previous enrollment in clinical trials, and hemolyzed plasma samples. All healthy control subjects were screened for pertinent oncologic and neurologic medical histories. Individuals with a history of cancer, neurological disorders, and infectious diseases were excluded from the study. All samples were collected with written informed consent after the patient was advised of the potential risks and benefits, as well as the investigational nature of the study. Our studies were conducted in accordance with principles for human experimentation as defined in the U.S. Common Rule and was approved by the Human Investigational Review Board of each study center under Partners institutional review board (IRB)—approved protocol number 2017P001581. BRISQ guideline reports are included in Table 2. Samples are taken from patient population undergoing treatment at MGH, HFHS, or WUStL. ExoLution PLUS is a proprietary kit available from Exosome Diagnostics (a BioTechne brand).

TABLE 1
Patient Demographics. IQR (Interquartile Range).
N/A = Not applicable.

| | | | | | Blinded | |
|---|---|---|---|---|---|---|
| | | | | | Multi- | |
| | | | | | Institution | |
| | Discovery | | Discovery | | Validation | |
| | Set 1 | % | Set 2 | % | Cohort | % |
| Total | 35 | | 48 | | 74 | |
| Disease | 50 | | 59.5 | | 56 | |
| Group Age | (43-64) | | (50-68.5) | | (46-66.5) | |
| median, IQR | | | | | | |
| Healthy | 31.5 | | 26.5 | | 55.5 | |
| Group Age | (26-52.75) | | (24-29.75) | | (48.5-63.3) | |
| median, IQR | | | | | | |
| Non-Tumor | N/A | | N/A | | 66 | |
| Group Age | | | | | (62-67) | |
| median, IQR | | | | | | |

Sex

| | | | | | | |
|---|---|---|---|---|---|---|
| Male | 16 | 45.7 | 25 | 52.1 | 46 | 62.2 |
| Female | 19 | 54.3 | 23 | 47.9 | 28 | 37.8 |

Race, n (%)

| | | | | | | |
|---|---|---|---|---|---|---|
| White | 31 | 88.6 | 43 | 89.6 | 65 | 87.8 |
| Asian | 1 | 2.9 | 2 | 4.2 | 3 | 4.1 |
| Black | 0 | 0.0 | 0 | 0.0 | 3 | 4.1 |
| Other | 2 | 5.7 | 1 | 2.1 | 0 | 0.0 |
| Unavailable | 1 | 2.9 | 2 | 4.2 | 3 | 4.1 |

Ethnicity, n (%)

| | | | | | | |
|---|---|---|---|---|---|---|
| Hispanic | 1 | 2.9 | 2 | 4.2 | 0 | 0.0 |
| Non-Hispanic | 30 | 85.7 | 40 | 83.3 | 55 | 74.3 |
| Unavailable | 4 | 11.4 | 6 | 12.5 | 19 | 25.7 |

WHO Grade, n (%)

| | | | | | | |
|---|---|---|---|---|---|---|
| II | 3 | 8.6 | 2 | 4.2 | 2 | 2.7 |
| III | 3 | 8.6 | 4 | 8.3 | 10 | 13.5 |
| IV | 16 | 45.7 | 32 | 66.7 | 33 | 44.6 |
| N/A | 13 | 37.1 | 10 | 20.8 | 29 | 39.2 |

Disease Status

| | | | | | | |
|---|---|---|---|---|---|---|
| Newly | 21 | 60.0 | 30 | 62.5 | 39 | 52.7 |
| Diagnosed | | | | | | |
| Recurrent | 4 | 11.4 | 8 | 16.7 | 18 | 24.3 |
| N/A | | | | | 17 | 23.0 |

Medications, n (%)

| | | | | | | |
|---|---|---|---|---|---|---|
| anticonvulsants | 16 | 45.7 | 13 | 27.1 | 30 | 40.5 |
| (levetriacetam, | | | | | | |
| clonazepam, | | | | | | |
| lacosomide, | | | | | | |
| carbamazepine, | | | | | | |
| lamotrigine) | | | | | | |
| steroids | 7 | 20.0 | 10 | 20.8 | 18 | 24.3 |
| (dexamethasone) | | | | | | |
| blood thinner | 3 | 8.6 | 4 | 8.3 | 9 | 12.2 |
| (aspirin, | | | | | | |
| coumadin) | | | | | | |
| prior radiation | 1 | 2.9 | 5 | 10.4 | 2 | 2.7 |
| therapy | | | | | | |
| prior | 2 | 5.7 | 8 | 16.7 | 4 | 5.4 |
| chemotherapy | | | | | | |
| clinical trial | 1 | 2.9 | 1 | 2.1 | 6 | 8.1 |
| participant | | | | | | |

TABLE 2
BRISQ reporting guidelines for study cohort.
I. Pre-acquisition:

| | |
|---|---|
| Biospecimen type: | Solid tissue, plasma |
| Anatomical Site: | Brain tumor tissue from disease site, Antecubital arm for |
| | peripheral blood |
| Disease status: | Specimens were obtained from adults with known or suspected |
| | gliomas who had no prior history of other primary tumors or |
| | active infections. Samples were also obtained from adult |
| | healthy controls with no prior history of cancer, neurological |
| | disorders, or active infections. |
| Clinical characteristics of patients: | Pertinent clinical data obtained were age, sex, tumor location, |
| | tumor volume, tumor pathology (histological and molecular |
| | features), time since onset of symptoms, and prior oncology |
| | treatment, if applicable. Clinical data for healthy controls |
| | included age, sex, and current medications. |
| Vital State: | All samples were collected from live patients. |
| Diagnosis: | For glioma patients, all diagnoses were based on tumor tissue |
| | pathological distinctions defined by a neuropathologist. Healthy |
| | controls were determined by a self-reported medical evaluation |
| | and basic chemistry blood tests. Pathology included TERT |
| | mutant glioma, TERT wildtype glioma, and healthy control. |

II. Acquisition:

| | |
|---|---|
| Collection mechanism and parameters: | Fresh tumor tissue obtained during surgical resection was |
| | collected in sterile containers with saline and kept in the |
| | operating room until completion of the surgical procedure. |
| | Tissue specimen were flash frozen with or without RNALater |
| | immediately. Whole blood was collected via standard |
| | venipuncture or obtained from arterial lines prior to surgical |
| | tumor tissue resection. All healthy control plasma samples were |
| | collected from a standard venipuncture. |
| Time from biospecimen | All biospecimen were processed within 2 hours of collection. |
| excision/acquisition to stabilization: | |

III. Stabilization/Preservation:

| | |
|---|---|
| Mechanism of stabilization: | RNA Later was used to preserve tumor tissue. K2 EDTA tubes |
| | were used to collect whole blood. No stabilization reagent was |
| | used for processed plasma. All samples were processed at room |
| | temperature. |
| Type of long-term preservation: | All samples were stored at −80° C. |

IV. Storage/Transport

| | |
|---|---|
| Storage temperature: | All biospecimen were collected at room temperature and were |
| | stored at −80° C. following processing. |
| Storage duration: | 1 month to 2 years. |

V. Quality Assurance Measures Relevant to the Extracted Product and Processing Prior to Analyte
Extraction and Evaluation:

| | |
|---|---|
| Composition assessment and selection: | Tumor presence in extracted tissue was determined by a |
| | neuropathologist. Plasma samples were evaluated for hemolysis |
| | based on color and hemolysed samples were excluded from this |
| | study. |

Tumor tissue processing. Tumor tissue was microdissected and suspended in RNAlater (Ambion) or flash-frozen and stored at −80° C.

Patient plasma processing. Whole blood was collected using K2 EDTA tubes with an inert gel barrier (BD Vacutainer Blood Collection Tubes), from pre-operatively placed arterial lines or venipuncture. Within 2 hours of collection, samples were centrifuged at 1,100×g for 10 minutes at 20° C. to separate the plasma from the hematocrit and filtered using 0.8 μm filters. 1 ml aliquots were stored at −80° C. for later downstream analysis. With the exception of the longitudinal samples, all baseline samples were collected prior to surgical resection.

Cell lines. Human carcinoma cell line A431 (ATCC CRL-1555) was cultured in Dulbecco's modified essential medium with high glucose (DMEM; Gibco, Invitrogen Cell Culture), containing 10% fetal bovine serum (FBS; Life Technologies Corporation) and 1% Penicillin/Streptomycin (Life Technologies). Human glioma cell line U87 (ATCC HTB-14) was cultured in DMEM with high glucose, containing 10% FBS and 1% Penicillin/Streptomycin. Human brain microvascular endothelial cells (HBMVEC) were kindly provided by Xandra O. Breakefield and cultured using endothelial basal medium (EGM-2 MV Microvascular Endothelial Cell Growth Medium-2 BulletKit, Lonza). All cell lines were grown to 50-70% confluency prior to gDNA extraction to minimize cell death and optimize quality of gDNA. All cell lines were verified monthly for mycoplasma contamination using commercial mycoplasma PCR (Mycoplasma PCR Detection Kit, Applied Biological Materials) to ensure lack of mycoplasma contamination.

DNA isolation. DNA was isolated from cell lines and frozen tumor tissue using the DNeasy Blood and Tissue Kit (Qiagen) as recommended by the manufacturer. DNA was eluted in AE buffer (Qiagen) and stored at −20° C. until further processing. DNA concentration and purity were determined using the NanoDrop One (ThermoFisher Scientific).

Plasma cfDNA isolation. Circulating nucleic acid was extracted from plasma using the QIAamp Circulating Nucleic Acid Kit (Qiagen) or ExoLution PLUS (Exosome Diagnostics) as per the manufacturer's instructions. cfDNA eluted in 20 μL AVE buffer (QIAamp Circulating Nucleic Acid Kit) or in 20 μL nuclease-free water (ExoLution PLUS Kit) and stored at −20° C. until quantification and subsequent ddPCR test.

TERT ddPCR assay. Since both the C228T and C250T TERT promoter mutations yield the same sequence (FIG. 1B), a single probe was used to detect both mutations. A second probe was also used to recognize the C228 wild type locus. As described by McEvoy et al., Locked Nucleic Acid (LNA) modifications were introduced on probes due to the short size of the probe, indicated by “+” (McEvoy et al., Sensitive droplet digital PCR method for detection of TERT promoter mutations in cell free DNA from patients with metastatic melanoma. Oncotarget 8, 78890-78900 (2017)). The sequences for the probes used are as follows: TERT promoter mutant (5′-FAM/CCC+C+T+T+CCGG (SEQ ID NO: 3)/3IABkFQ/) and TERT promoter wild type (5′-HEX/CCC C+C+T+CCG G (SEQ ID NO: 4)/3IABkFQ/). Probes were synthesized by Integrated DNA Technologies (IDT). ddPCR amplification was performed using either 4 μL of cfDNA template or using 100 ng of tumor gDNA, 1× ddPCR Supermix for probes (no dUTP, Bio-Rad), either 1× Q-sol or 200 mM 7-deaza-dGTP (7dG; New England Biolabs), 250 nM of each probe and 900 nM of each primer (5′-CCTGCCCCTTCACCTTCCAG-3′ (SEQ ID NO: 1) and 5′-AGAGCGGAAAGGAAGGGGA-3′) (SEQ ID NO: 2) with template (100 ng of tumor tissue or 4 μL of cfDNA) in a total reaction mix of 20 μL. The QX200 manual droplet generator (Bio-Rad) was used to generate droplets. Thermocycling conditions were as follows: 95° C. (51% ramp) for 10 minutes, 40 cycles of 94° C. (51% ramp) for 30 seconds and 57° C. for 1 minute, followed by 98° C. for 10 minutes and held at 4° C. until further processing. Droplets were counted and analyzed using the QX200 droplet reader (Bio-Rad) and QuantaSoft analysis (Bio-Rad) was performed to acquire data.

Quantification of Copies/mL of Plasma. Copies per mL of plasma is calculated by taking copies/204, (C; provided by QuantaSoft), multiplied by elution volume in μL (EV), divided by the total volume added to the reaction (TV), divided by the plasma volume (PV). In short, Copies/mL of plasma=C*EV/TV/PV.

R-based ddPCR Analysis of MAF from Plasma Samples. Gates were constructed for both channel 1 (4-8) and channel 2 (2-6) in increments of 0.05. Combinations of channel 1 gates and channel 2 gates were used to calculate MAF, the number of channel 1 positive droplets divided by the number of channel 2 positive droplets. Thresholds were calculated to fulfill one of the following criteria: maximize specificity, set specificity close to 90%, and to minimize the distance from the ROC curve to the point (0,1). These gating strategies (trained using a discovery cohort) were then used to analyze the data from the multi-institution validation cohort in a blinded fashion. Code available on GitHub Repository koushikmuralidharan/BILL.

Statistical Analysis. Statistical analyses were performed using unpaired two-tailed Student's t-test in GraphPad Prism 8 software and p<0.05 was considered statistically significant. Confidence intervals were calculated using exact binomial distributions. The results are presented as the mean±SD. “***” indicates p-value less than or equal to 0.001, and “****” indicates p-value less than or equal to 0.0001.

### Example 1: Assay Design and Optimization

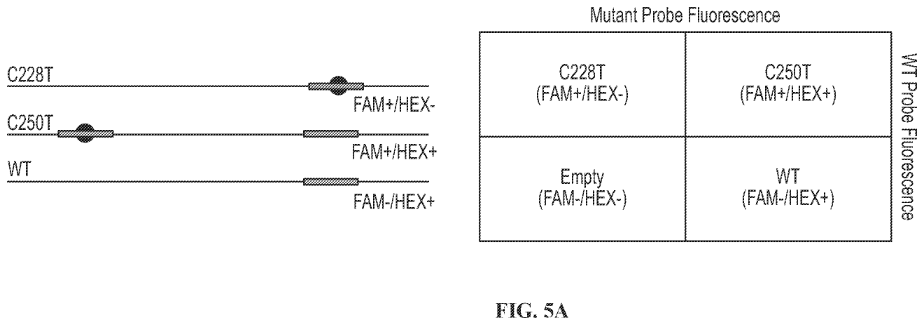

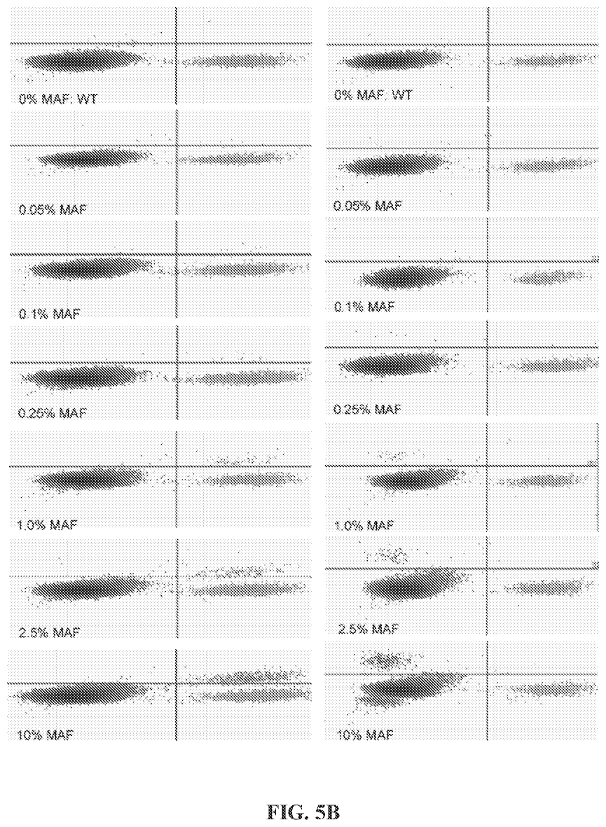

The most common TERT promoter mutations, C228T and C250T, are heterozygous and mutually exclusive, but both mutations result in the generation of an 11-bp identical sequence, 5′-CCCCTTCCGGG-3′ (SEQ ID NO: 5). A 10-bp LNA mutant probe was used to simultaneously detect both mutations and an LNA wild-type probe complementary to the C228 locus (FIG. 1B) was used to detect wild-type DNA. Assay specificity for each mutation was established using U87 (C228T mutant), A431 (C250T mutant), and HBMVEC (TERT WT) gDNA (FIG. 1C). For inputs with mutant allele frequencies (MAF) greater than 10%, 2D amplitude analysis can distinguish between C250T/C228T mutations (FIG. 1D; FIGS. 5A-5B). TERT promoter mutations are situated in a GC-rich region resistant to amplification that hinders assay performance. To stabilize amplification, Q-sol additive was compared to 7-deaza-dGTP (7dG), a modified nucleotide that inhibits secondary structure formation (Motz et al., Improved Cycle Sequencing of GC-Rich Templates by a Combination of Nucleotide Analogs. BioTechniques vol. 29 268-270 (2000)). TERT assay performance with 7dG was superior to Q-Sol (1.8-3.9-fold increase, p=0.04) with higher absolute mutant detection and comparable WT detection (FIGS. 1E-1F). Analytical parameters including limit-of-detections (LODs) of 0.27% MAF (C250T) and 0.42% (C228T) MAF and limit of blanks of 0.02% (C250T) and 0.04% MAF (C228T; FIGS. 1G-1H; FIG. 5B) are reported herein. In a comparison of extraction platforms to optimize TERT recovery, a two-fold increase in TERT WT (p=0.001) was seen while using ExoLution PLUS in comparison to the QiAmp Circulating Nucleic Acid kit (Qiagen) (FIG. 1I). Two mL of plasma was determined to be the optimal input for recovery as a function of copies of TERT WT per mL of plasma (FIGS. 1J-1K).

Taken together, these results demonstrate detection of TERT promoter mutations using ddPCR assays described herein.

### Example 2: Development of R-Based Analysis for TERT Promoter ddPCR Analysis and Cohort Design

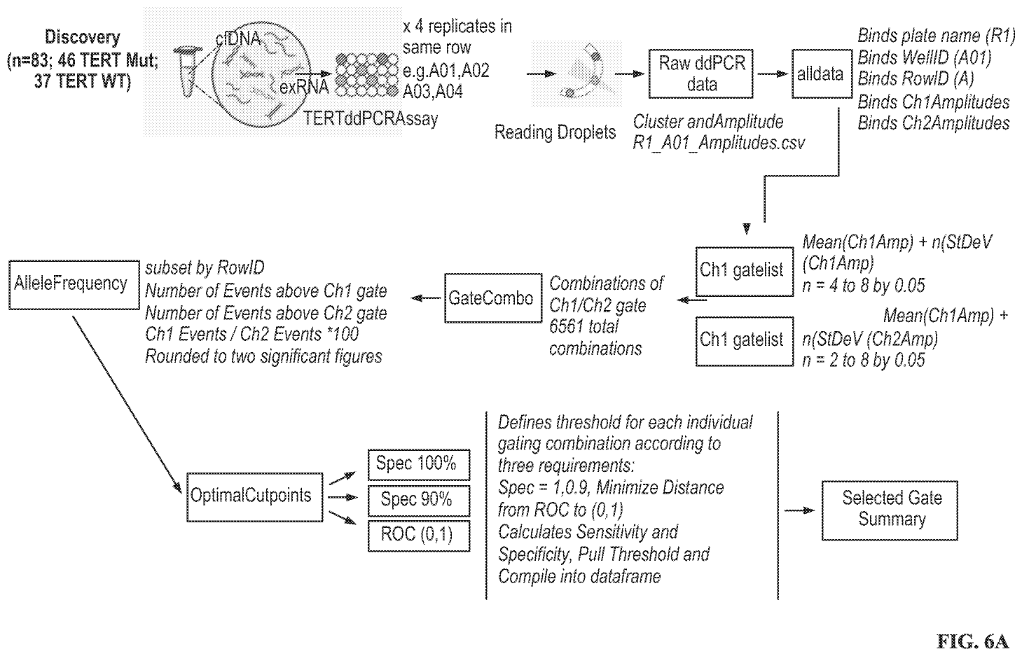

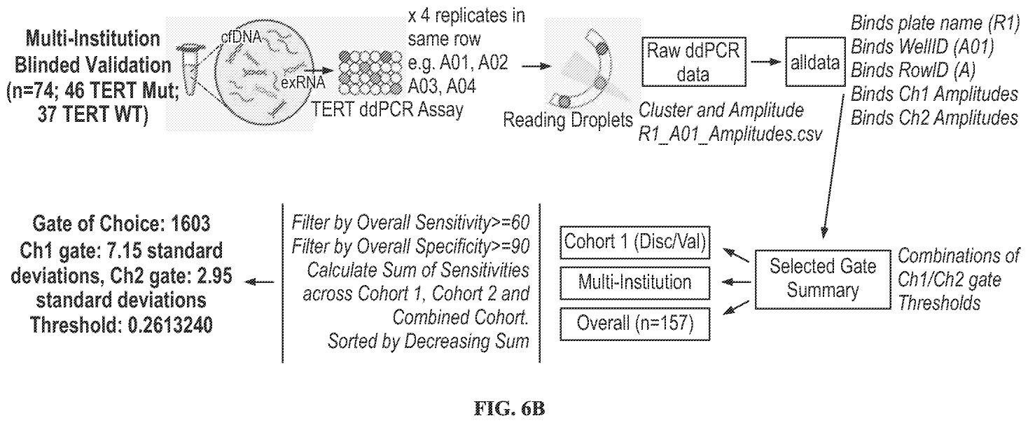

To standardize gating without operator bias, it was sought to mathematically define and automate the gating strategy. The gating strategy described herein is based on the algorithm used by the R-program ddPCR, which defines empty droplets as those that lie within 7 standard deviations above the mean amplitude of channel 1 (Corless et al., Development of Novel Mutation-Specific Droplet Digital PCR Assays Detecting TERT Promoter Mutations in Tumor and Plasma Samples. J. Mol. Diagn. 21, 274-285 (2019)). To determine the optimal gating strategy, experiments were gated continuously from 4-8 standard deviations above the mean amplitude of channel 1 and 2-6 standard deviations above the mean amplitude of channel 2 in increments of 0.05, with pseudocode provided in FIGS. 6A-6B. Bootstrapping with 1000 replicates was used to determine a threshold, and a list of gating strategies with threshold values was generated to ensure that specificity was greater than or equal to 90%. One such gating strategy that maximizes the sum of discovery sensitivity, validation sensitivity, and overall sensitivity is described herein. This gating strategy gates at 7.15 standard deviations above the mean of channel 1 amplitude, 2.95 standard deviations above the mean of channel 2 amplitude, with a threshold of 0.26% MAF (FIGS. 6A-6B). This is in accordance with prior literature, which suggests that positive droplets lie 7 standard deviations above the mean amplitude of channel 1 (Corless et al., Development of Novel Mutation-Specific Droplet Digital PCR Assays Detecting TERT Promoter Mutations in Tumor and Plasma Samples. J. Mol. Diagn. 21, 274-285 (2019)). Notably, the threshold calculated by the program, 0.26% MAF, was equivalent to the experimentally determined LOD (MAF) of the TERT Promoter ddPCR Assay using cell line derived gDNA (FIG. 1G).

Taken together, these results report a gating strategy that maximizes the sum of discovery sensitivity, validation sensitivity, and overall sensitivity.

### Example 3: Detection of TERT Promoter Mutations in cfDNA from Discovery and Blinded Validation Cohort

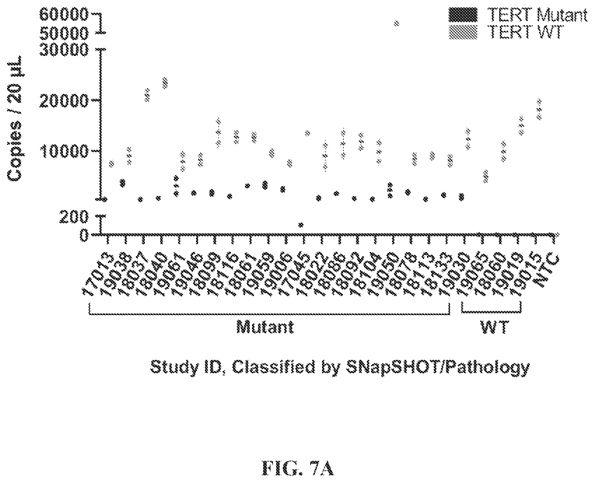

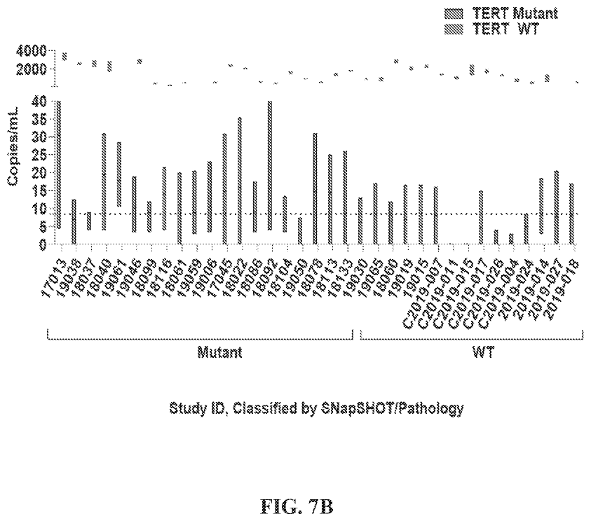

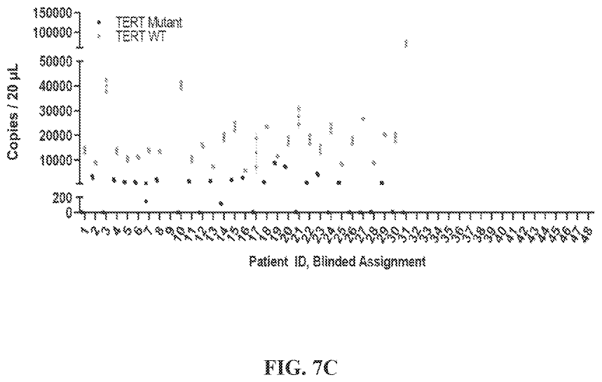

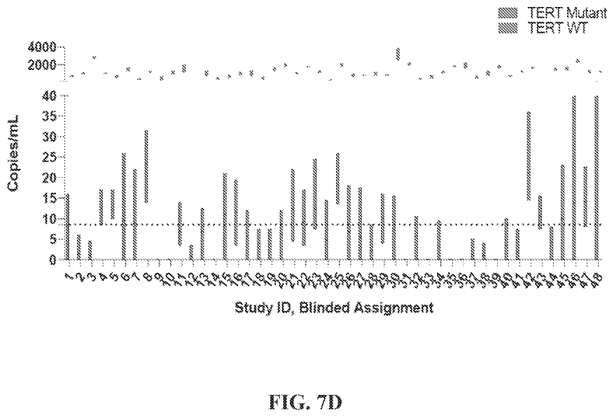

A cohort (n=157) of molecularly characterized glioma patients (n=114), non-tumor patients with enhancing lesions on MRI (n=9), and age matched healthy controls (n=33) were selected. The patient population spanned tumor diagnosis (20% astrocytoma, 7% oligodendroglioma, 72% glioblastoma, 1% gliosarcoma); grade (6% II; 15% III, 71% IV, 8% not reported) and molecular characteristics: IDH1 mutant (19%), TERT mutant (45% C228T; 15% C250T), 1p19q codeletion (7%), EGFR amplified (20%), MGMT methylated (33%) (Table 1; FIGS. 2A-2E). The study population was randomly assigned to either a discovery cohort (n=83) or a blinded multi-institutional validation cohort (n=74). To assess the clinical performance of the assay, tumor tissue and matched plasma samples were analyzed for the presence of the TERT mutations (FIGS. 7A-7D).

In tissue, it was demonstrated that the assay described herein and the CLIA certified Solid SNAPSHOT assay (Attali et al., ddper: an R package and web application for analysis of droplet digital PCR data. F1000Research vol. 5 1411 (2016)) used in the MGH Department of Pathology, detected TERT positive tumors with 100% concordance across 97 tested samples for the presence of the TERT promoter mutation. (FIG. 2A, FIGS. 7A-7D). In one patient, using parallel tumor tissue aliquots, our assay detected the C250T mutation while the SNAPSHOT assay detected the C228T mutation.

Plasma samples (Total n=83; TERT mutant n=46; TERT WT n=17; healthy controls n=20) were analyzed and gated (FIGS. 6A-6B) in two separate discovery sample sets (FIG. 2A). In sample set 1, studies described herein report positivity in 16 of 21 (76%) TERT mutant samples, and in 1 of 14 (7%) WT control samples (FIG. 2B), with a sensitivity of 76.19% (CI, 52.83-91.78), and specificity of 92.86% (CI, 66.3-99.82). In sample set 2 (PCR blinded), studies described herein report positivity in 17 of 25 (68%) of TERT mutant samples, and in 3 of 20 (15%) of WT control samples (FIG. 2C), with a sensitivity of 68.00% (CI, 46.50-85.05), and specificity of 86.96% (CI, 66.41-97.22). Overall, in the discovery cohort, studies described herein report positivity in 33 of 46 (72%) TERT mutant samples, and in 4 of 37 (11%) TERT WT samples (FIG. 2D), with a sensitivity of 71.74% (CI, 56.54-84.01) and specificity of 89.19% (CI, 74.58-96.97).

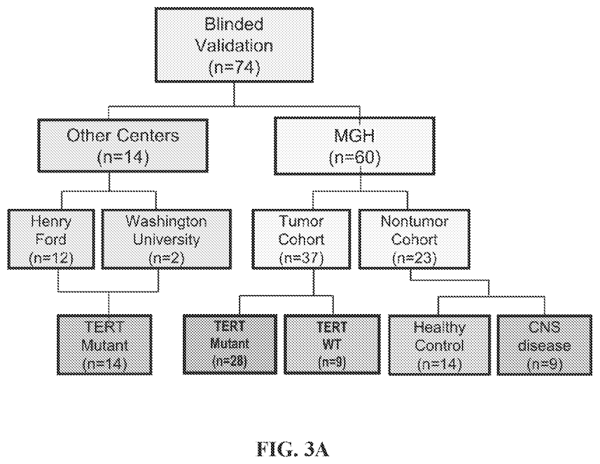

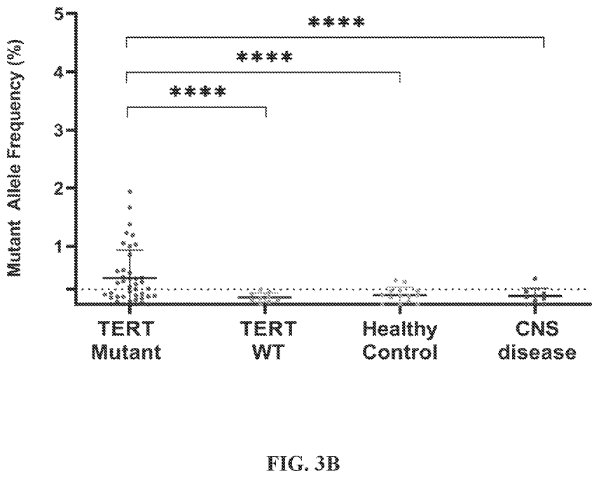

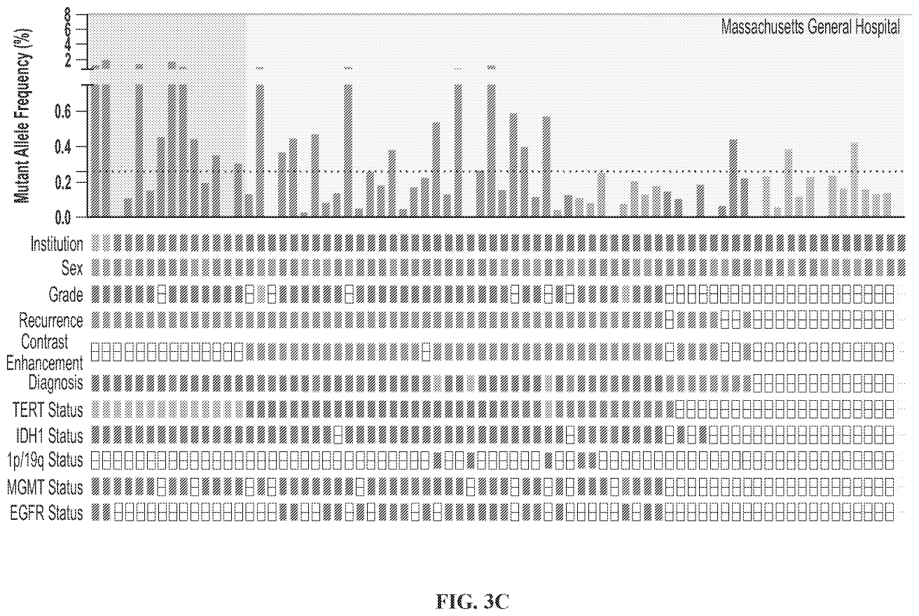

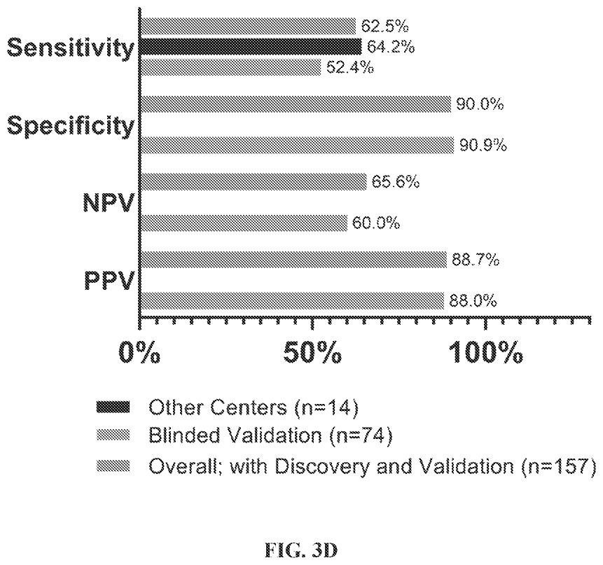

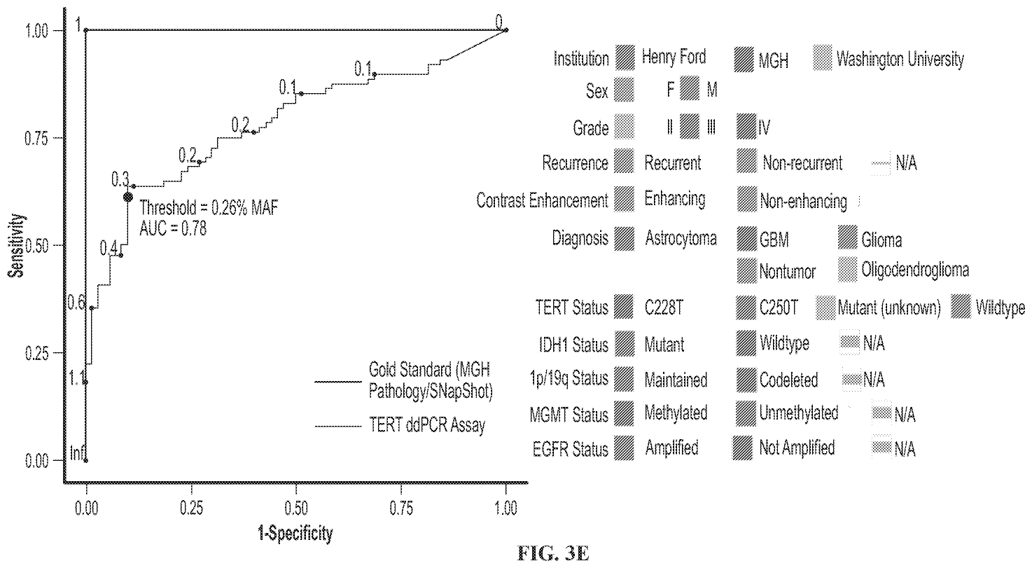

To validate assay performance in multi-institutional samples, a blinded cohort (Total n=74; TERT mutant n=41 (Henry Ford Hospital n=12; Washington University n=2; Massachusetts General Hospital n=27); TERT WT n=17; healthy control n=14, CNS disease non-glioma n=9) was analyzed using the parameters previously described (FIG. 3A, FIGS. 6A-6B). The CNS disease non-tumor samples were from patients who either had a non-malignant contrast enhancing mass on MRI, such as a demyelinating lesion or fungal abscess, which was initially suspected as glioma or other non-tumor conditions such as normal pressure hydrocephalus. In matched plasma, studies described herein report positivity in 22 of 42 (52%) confirmed mutant samples and 3 of 33 (9%) confirmed wild-type with a sensitivity of 52.38% (CI, 36.42%-68.00%), specificity of 90.91% (CI, 75.67% to 98.08%) (FIGS. 3B-3D).

The blinded multi-institution validation cohort verifies and validates our assay performance for the detection of the TERT promoter mutation in plasma (Juratli et al., Intratumoral heterogeneity and promoter mutations in progressive/higher-grade meningiomas. Oncotarget 8, 109228-109237 (2017); Nakajima et al., BRAF V600E, TERT promoter mutations and CDKN2A/B homozygous deletions are frequent in epithelioid glioblastomas: a histological and molecular analysis focusing on intratumoral heterogeneity. Brain Pathol. 28, 663-673 (2018); and Sottoriva et al., Intratumor heterogeneity in human glioblastoma reflects cancer evolutionary dynamics. Proc. Natl. Acad. Sci. 110, 4009-4014 (2013)). In summary, combining all three cohorts (n=157), a sensitivity of 62.50% (CI, 51.53%-72.60%), and a specificity of 90% (CI, 80.48%-95.8%) was demonstrated (FIG. 2E and FIG. 3D). Of patients who were classified positive by plasma analysis but negative by tissue analysis at the MAF threshold selected, the clinical scenario of these false positive samples included 4/34 healthy controls, 1/9 patients with non-glioma CNS disease, and 2/34 TERT WT gliomas.

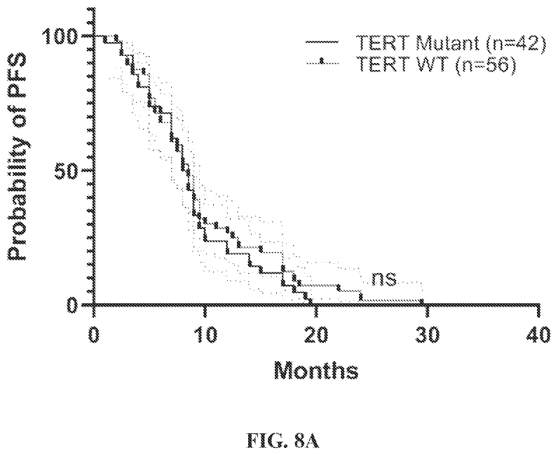

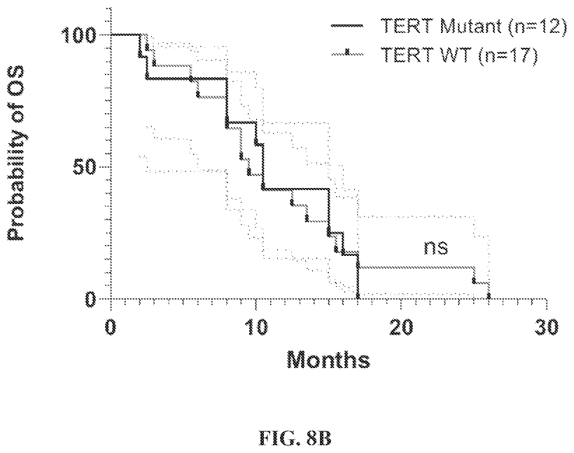

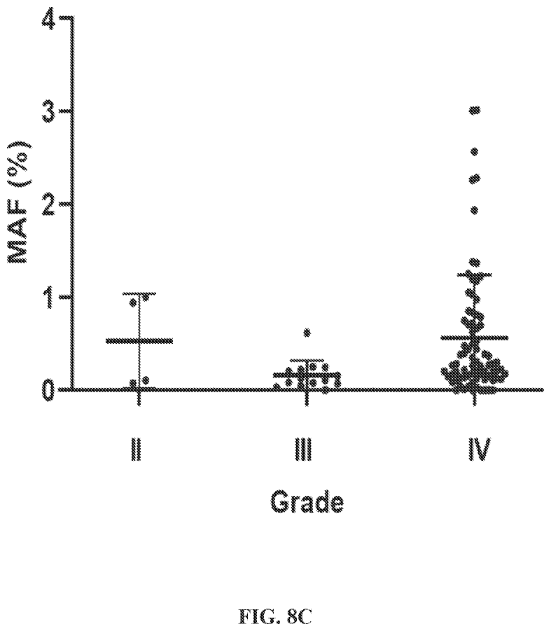

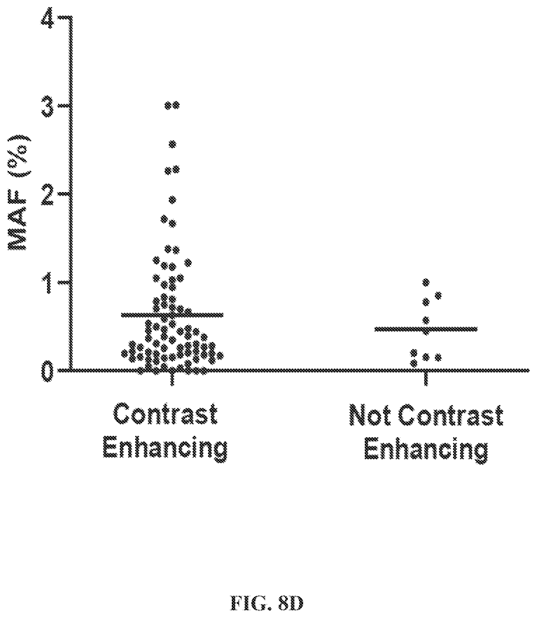

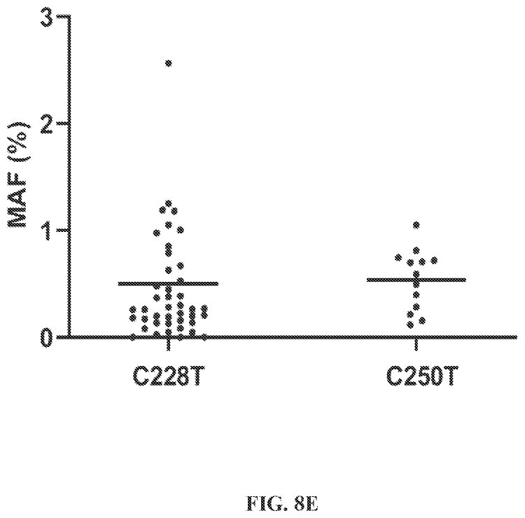

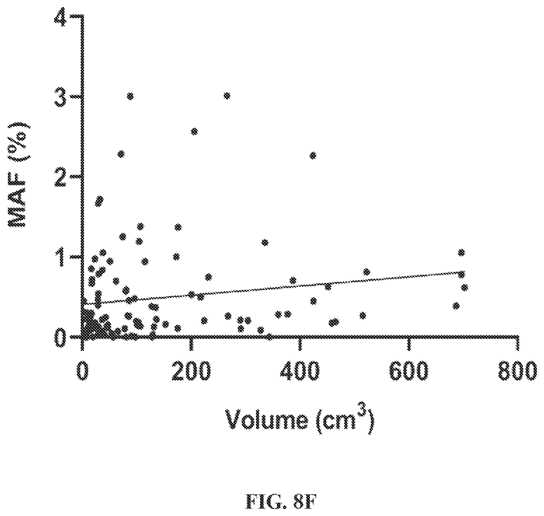

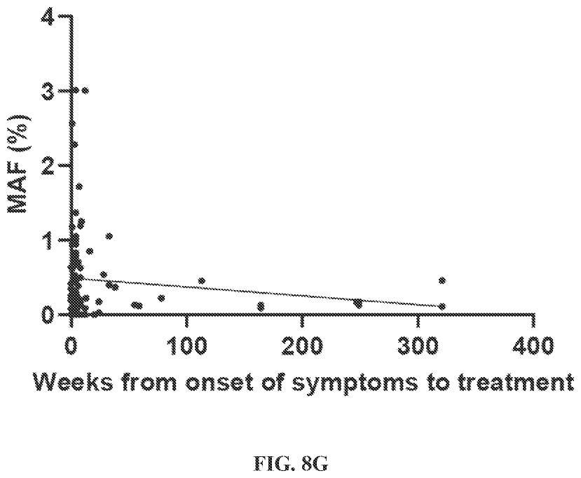

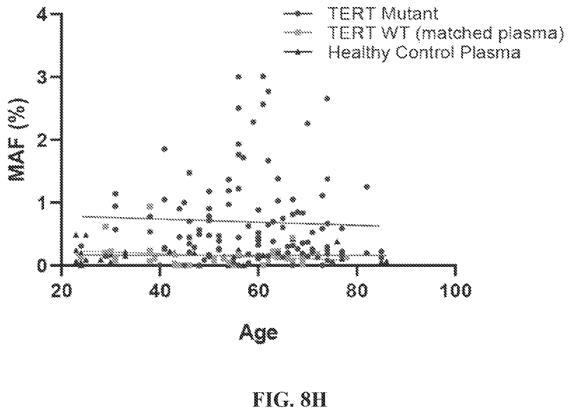

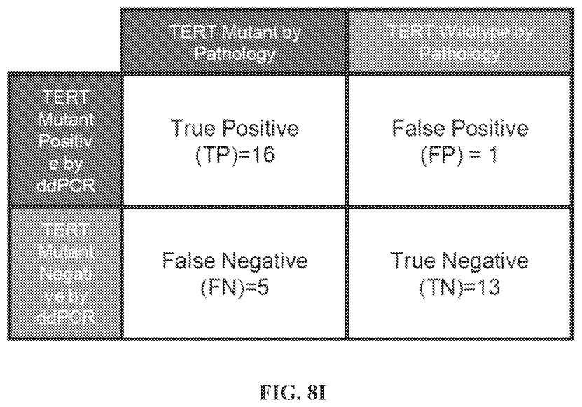

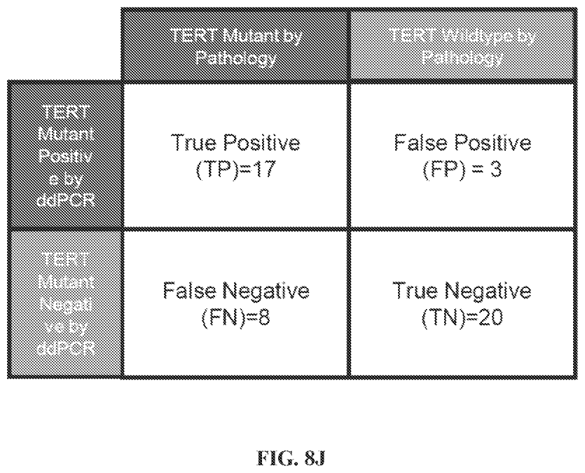

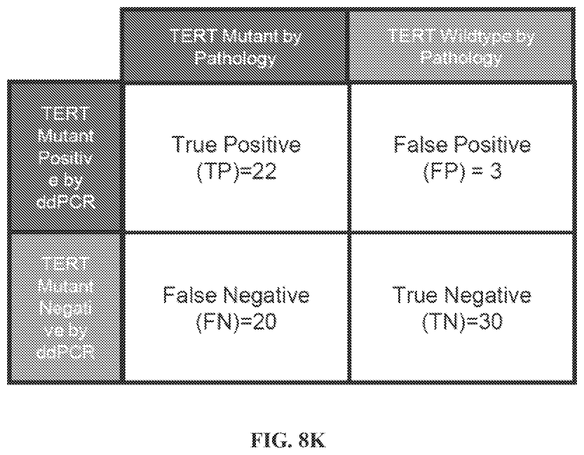

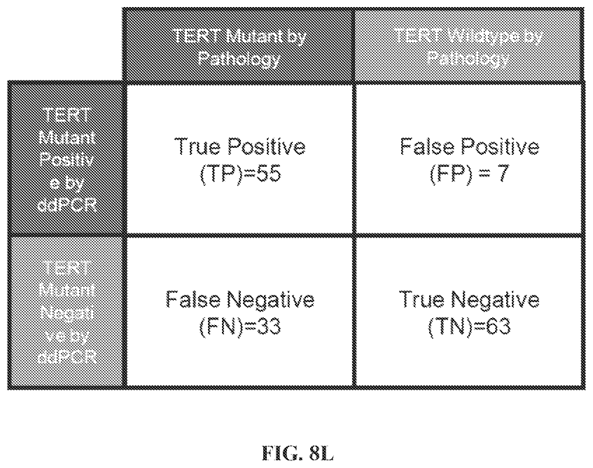

Within the limits of our cohort, no significant correlation was detected between TERT MAF in plasma and age, duration of symptoms, tumor grade, mutational status (C228T/C250T), tumor volume, contrast enhancement, overall survival and progression free survival (FIGS. 8A-8L). However, it was observed that patients with contrast enhancing tumors (increased breakdown of blood brain barrier) tended to have higher MAF than patients with non-contrast enhancing tumors (FIG. 8G). Furthermore, patients with MAF above threshold tended to have poorer progression free survival and overall survival compared to patients with below threshold MAF (FIGS. 8A-8B).

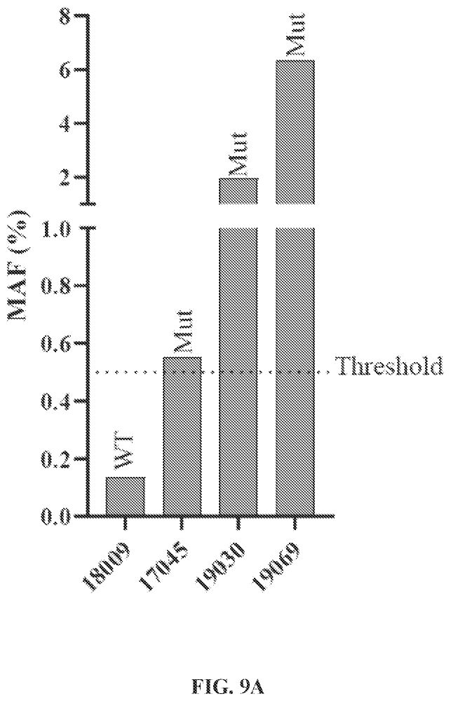

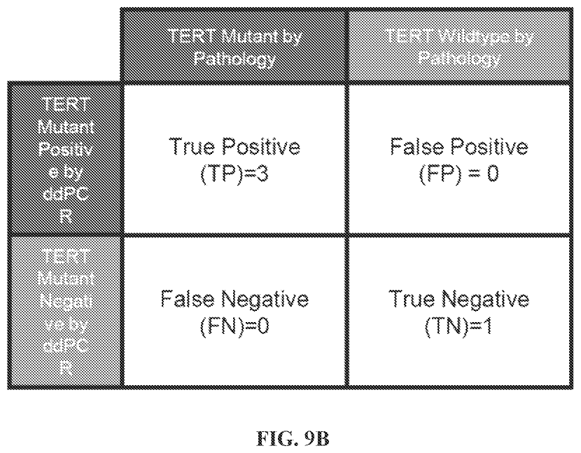

Also reported herein is perfect concordance in 4 available matched CSF samples from both cohorts compared to a tissue gold standard (TERT mutant n=3; TERT WT n=1). A TERT mutation was also detected in the CSF of a patient sample whose plasma TERT MAF was below the defined assay threshold (FIGS. 9A-9B).

Taken together, these results demonstrate detection of TERT promoter mutation in plasma of a discovery cohort and a blinded multi-institution validation cohort.

TABLE 3
Patient Characteristics for Discovery Cohort 1. Tumor volume calculated by taking three measurements and using the following formula:
4pi/3 * R1 * R2 * R3. GBM = Glioblastoma, WT = Wildtype, N/A = Not Available, M = Male, F = Female

| | | | | | | | | | | | | TERT | Copies/20 μL of | Copies/mL of | Copies/mL of |
|---|---|---|---|---|---|---|---|---|---|---|---|---|---|---|---|
| | | | | | | | | | | | TERT | Status by | TERT Mutant | TERT Mutant | TERT WT |
| Study | | | WHO | | IDH1 | | Volume | Contrast | Duration | | Status | ddPCR | from Tumor | from ddPCR of | from ddPCR of |
| ID | Age | Sex | Grade | Diagnosis | status | Location | (cm3) | Enhancement | (weeks) | Recurrent | (MGH) | (Tissue) | Tissue ddPCR | Matched Plasma | Matched Plasma |
| MGH- | 24 | M | III | Recurrent | IDH1 | L frontal | 3.015929 | YES | N/A | YES | C228T | C228T | 1720 | 10.25 | 2941.25 |
| 19046 | | | | Oligodendroglioma | R132H | | | | | | | | | | |
| BC- | 67 | M | IV | GBM | WT | R frontotemporal | 696.5372 | YES | 4 | NO | C250T | C250T | 303 | 7.25 | 2598.75 |
| 18037 | | | | | | | | | | | | | | | |
| BC- | 48 | F | IV | GBM | WT | R frontal | 217.7752 | YES | 4 | NO | C250T | C250T | 522 | 19.375 | 2473.75 |
| 18040 | | | | | | | | | | | | | | | |
| MGH- | 61 | M | IV | GBM | WT | L temporal | 265.8541 | YES | 8 | NO | C228T | C228T | 3103 | 17.75 | 4750 |
| 19061 | | | | | | | | | | | | | | | |
| MGH- | 49 | M | III | Anaplastic | IDH1 | R mesial temporal | 327.6807 | NO | 4 | NO | C228T | C228T | 3120 | 11.125 | 508.75 |
| 18061 | | | | Astrocytoma | R132S | | | | | | | | | | |
| BC- | 57 | M | N/A | GBM | WT | R temporal | 32.75634 | YES | N/A | YES | C228T | C2287 | 431 | 30.5 | 3448.75 |
| 17013 | | | | | | | | | | | | | | | |
| MGH- | 29 | F | II | Oligodendroglioma | IDH1 | R frontal | 14.70265 | NO | 7 | NO | C228T | C228T | 1035 | 14.125 | 222.5 |
| 18116 | | | | | R132H | | | | | | | | | | |
| MGH- | 46 | F | N/A | Recurrent | IDH1 | L temporal | 424.869 | NO | N/A | YES | C250T | C250T | 1682 | 8.875 | 427.5 |
| 18099 | | | | Oligodendroglioma | R132H | | | | | | | | | | |
| MGH- | 71 | M | IV | GBM | WT | L anterior temporal | 267.7475 | YES | 4 | NO | Unknown | C228T | 2416 | 10.375 | 547.5 |
| 19006 | | | | | | | | | | | | | | | |
| MGH- | 77 | M | IV | Diffuse Astrocytoma | WT | L temporal/occipital | N/A | N/A | 4 | NO | C228T | C228T | 3260 | 8.875 | 78.75 |
| 19059 | | | | | | | | | | | | | | | |
| MGH- | 54 | F | IV | GBM | WT | R temporoparietal | 23.49911 | YES | 3 | NO | C228T | C228T | 3630 | 7 | 2548.75 |
| 19038 | | | | | | | | | | | | | | | |
| MGH- | 38 | F | II | Oligodendroglioma | IDH1 | R frontoparietal | 696.6796 | YES | 3 | NO | C250T | C250T | 1593 | 9.125 | 600 |
| 18086 | | | | | R132H | | | | | | | | | | |
| BC- | 46 | F | IV | GBM | WT | R frontoparietal | 387.4631 | YES | 4 | NO | C250T | C250T | 653 | 15.85714 | 2057.143 |
| 18022 | | | | | | | | | | | | | | | |
| BC- | 47 | F | IV | GBM | IDH1 | R temporal | 377.3681 | YES | 6 | NO | C250T | C250T | 104 | 14.85714 | 2350 |
| 17045 | | | | | R132C | | | | | | | | | | |
| MGH- | 63 | M | IV | GBM | WT | L temporoparietal | 61.92707 | YES | 4 | NO | C250T | C250T | 441 | 15.75 | 475 |
| 18092 | | | | | | | | | | | | | | | |
| MGH- | 52 | M | IV | GBM | WT | L frontal | 686.8276 | YES | 3 | NO | Unknown | C228T | 1826 | 14.75 | 588.75 |
| 18078 | | | | | | | | | | | | | | | |
| MGH- | 74 | F | IV | GBM | WT | L temporal | 16.9646 | YES | 6 | NO | C228T | C228T | 1229 | 8.625 | 1818.75 |
| 18133 | | | | | | | | | | | | | | | |
| MGH- | 41 | M | IV | GBM | WT | R temporal | 360.215 | YES | 3 | NO | C228T | C228T | 880 | 6.25 | 943.75 |
| 19030 | | | | | | | | | | | | | | | |
| MGH- | 82 | M | IV | GBM | WT | R parietal | 99.42512 | YES | 1 | NO | C228T | C228T | 415 | 14.375 | 1426.25 |
| 18113 | | | | | | | | | | | | | | | |
| MGH- | 50 | M | IV | GBM | WT | R occipital | 96.20813 | YES | 8 | NO | C228T | C228T | 2218 | 2.875 | 917.5 |
| 19050 | | | | | | | | | | | | | | | |
| MGH- | 74 | F | IV | Residual | WT | L anterior temporal | 23.12212 | YES | N/A | NO | C228T | C250T | 391 | 7.375 | 1602.5 |
| 18104 | | | | GBM | | | | | | | | | | | |
| MGH- | 30 | M | III | Anaplastic | IDH1 | R posterior | 136.1692 | NO | 8 | NO | WT | WT | 2.3 | 8.428572 | 870.0001 |
| 19065 | | | | Astrocytoma | R132G | | | | | | | | | | |
| MGH- | 64 | F | N/A | Recurrent/ | IDH1 | R frontal, multifocal | 8.385958 | YES | N/A | YES | WT | WT | 0 | 5.285715 | 2287.143 |
| 19015 | | | | Residual GBM | R132H | | | | | | | | | | |
| MGH- | 43 | M | IV | GBM | IDH1 | L frontal | 458.5804 | YES | 13 | NO | WT | WT | 0 | 6.428572 | 2851.429 |
| 18060 | | | | | R132H | | | | | | | | | | |
| MGH- | 39 | M | II | Diffuse | IDH1 | L parietal | 291.5398 | YES | 24 | NO | WT | WT | 1.8 | 7 | 2021.429 |
| 19019 | | | | Astrocytoma | R132H | | | | | | | | | | |
| C2019- | 30 | F | N/A | N/A | N/A | N/A | N/A | N/A | N/A | N/A | WT | N/A | N/A | 7.625 | 7075 |
| 027 | | | | | | | | | | | | | | | |
| C2019- | 24 | F | N/A | N/A | N/A | N/A | N/A | N/A | N/A | N/A | WT | N/A | N/A | 8.375 | 1121.25 |
| 014 | | | | | | | | | | | | | | | |
| C2019- | 25 | F | N/A | N/A | N/A | N/A | N/A | N/A | N/A | N/A | WT | N/A | N/A | 4.875 | 542.5 |
| 024 | | | | | | | | | | | | | | | |
| C2019- | 24 | M | N/A | N/A | N/A | N/A | N/A | N/A | N/A | N/A | WT | N/A | N/A | 0.75 | 731.25 |
| 004 | | | | | | | | | | | | | | | |
| C2019- | 33 | M | N/A | N/A | N/A | N/A | N/A | N/A | N/A | N/A | WT | N/A | N/A | 1 | 1308.75 |
| 026 | | | | | | | | | | | | | | | |
| C2019- | 43 | M | N/A | N/A | N/A | N/A | N/A | N/A | N/A | N/A | WT | N/A | N/A | 4.625 | 1707.5 |
| 017 | | | | | | | | | | | | | | | |
| C2019- | 56 | F | N/A | N/A | N/A | N/A | N/A | N/A | N/A | N/A | WT | N/A | N/A | 0 | 1890 |
| 015 | | | | | | | | | | | | | | | |
| C2019- | 86 | M | N/A | N/A | N/A | N/A | N/A | N/A | N/A | N/A | WT | N/A | N/A | 0 | 1030 |
| 011 | | | | | | | | | | | | | | | |
| C2019- | 77 | F | N/A | N/A | N/A | N/A | N/A | N/A | N/A | N/A | WT | N/A | N/A | 8 | 1400 |
| 007 | | | | | | | | | | | | | | | |
| C2019- | 29 | F | N/A | N/A | N/A | N/A | N/A | N/A | N/A | N/A | WT | N/A | N/A | 8 | 551.25 |
| 018 | | | | | | | | | | | | | | | |

TABLE 4
Patient Characteristics for Discovery Cohort 2. Tumor volume calculated by taking three measurements and using the following formula:
4pi/3 * R1 * R2 * R3. GBM = Glioblastoma, WT = Wild-type, N/A = Not Available, M = Male, F = Female

| | | | | | | | | | | | | | | Copies/mL of | Copies/mL of |
|---|---|---|---|---|---|---|---|---|---|---|---|---|---|---|---|
| | | | | | | | | | | | | TERT Status | | TERT Mutant | TERT WT |
| Blinded | | | WHO | | IDH1 | | Volume | Contrast | Duration | | TERT Status | by ddPCR | Mutant Allele | from ddPCR of | from ddPCR of |
| Cohort # | Age | Sex | Grade | Diagnosis | status | Location | (cm3) | Enhancement | (weeks) | Recurrent | (MGH) | (Tissue) | Frequency | Matched Plasma | Matched Plasma |
| 1 | 67 | F | IV | GBM | WT | Right multifocal | 13.57 | YES | 12 | NO | WT | WT | 6.7 | 8.38 | 713.75 |
| | | | | | | anterior temporal | | | | | | | | | |
| 2 | 56 | M | IV | GBM | WT | Right parietal | 97.64 | YES | 8 | NO | C228T | C228T | 3095 | 1.5 | 993.75 |
| 3 | 24 | M | IV | GBM | WT | Left parietal | 24.93 | YES | 12 | NO | WT | WT | 1.9 | 1.13 | 2815 |
| 4 | 64 | M | IV | GBM | WT | Left parietal and | 465.51 | YES | 20 | NO | C228T | C228T | 1911 | 12.63 | 1046.25 |
| | | | | | | posterior frontal | | | | | | | | | |
| 5 | 65 | M | IV | GBM | WT | Left parietal | 231.68 | YES | 4 | NO | C250T | C250T | 1089 | 12.13 | 666.25 |
| 6 | 46 | F | IV | GBM | WT | Right frontoparietal | 290.6 | YES | 2 | NO | C250T | C250T | 957 | 9.5 | 1487.5 |
| 7 | 67 | M | IV | GBM | WT | Right frontoparietal | 522.4 | YES | 3 | NO | C250T | C250T | 333 | 8.88 | 402.5 |
| 8 | 66 | M | IV | GBM | WT | Right parietal | 304.42 | YES | 2 | NO | C228T | C228T | 1927 | 23.38 | 1196.25 |
| 9 | 61 | M | IV | Recurrent/ | WT | Right frontal lobe | 7.35 | YES | 2 | YES | WT | N/A | N/A | 0 | 467.5 |
| | | | | Residual GBM | | | | | | | | | | | |
| 10 | 43 | M | IV | GBM | IDH1 | Left frontal | 343.94 | YES | 12 | NO | WT | WT | 1.8 | 0 | 1033.75 |
| | | | | | R132H | | | | | | | | | | |
| 11 | 52 | M | IV | GBM | WT | Left frontal | 515.12 | YES | 3 | NO | Unknown | C228T | 1370 | 6.75 | 1421.25 |
| 12 | 69 | M | IV | GBM | WT | Right frontal | 90.48 | YES | 3 | NO | WT | WT | 1.2 | 0.88 | 22350 |
| 13 | 74 | F | IV | Recurrent/ | WT | Left anterior temporal | 17.34 | YES | 4 | YES | C228T | C228T | 1512 | 6.88 | 970 |
| | | | | Residual GBM | | | | | | | | | | | |
| 14 | 50 | M | IV | GBM | WT | Right temporal | 335.67 | YES | 3 | NO | C228T | C228T | 116 | 0 | 536.25 |
| 15 | 82 | M | IV | GBM | WT | Right parietal | 74.57 | YES | 1 | NO | C228T | C228T | 1888 | 11.13 | 752.5 |
| 16 | 31 | M | II | Recurrent/ | IDH1 | Left parietal | 50.72 | YES | 9 | YES | Unknown | C228T | 2767 | 11.63 | 1061.25 |
| | | | | Residual | R132H | | | | | | | | | | |
| | | | | Oligodentroglioma | | | | | | | | | | | |
| 17 | 38 | M | III | Recurrent/ | IDH1 | Left posterior | 42.98 | YES | N/A | YES | WT | WT | 5.3 | 5.75 | 1023.75 |
| | | | | Residual | R132G | cingulum | | | | | | | | | |
| | | | | Infiltrating | | | | | | | | | | | |
| | | | | Astrocytoma | | | | | | | | | | | |
| 18 | 73 | F | IV | GBM | WT | Left parieto-occipital | 127.91 | YES | 1 | NO | C228T | C228T | 1037 | 4 | 495.5 |
| 19 | 71 | M | IV | GBM | WT | Left anterior temporal | 200.81 | YES | 4 | NO | C228T | C228T | 8710 | 3.88 | 1575 |
| 20 | 60 | F | IV | GBM | WT | Right frontal | 153.06 | YES | 3 | NO | C228T | C228T | 7350 | 6.25 | 1956.25 |
| 21 | 64 | F | IV | Recurrent/ | IDH1 | Right frontal | 11.08 | YES | 3 | YES | WT | WT | 4.4 | 12.38 | 1052.5 |
| | | | | Residual GBM | R132H | | | | | | | | | | |
| 22 | 61 | F | IV | GBM | WT | Right frontal | 205.84 | YES | 3 | NO | C228T | C228T | 764 | 9.13 | 1800 |
| 23 | 58 | M | IV | GBM | WT | Left frontotemporal | 451.89 | YES | 1 | NO | C228T | C228T | 4200 | 12.25 | 1252.5 |
| 24 | 43 | M | III | Recurrent/ | IDH1 | Middle frontal gyrus | 40.97 | YES | 8 | YES | WT | WT | 2.5 | 4.75 | 287.5 |
| | | | | Residual | R132H | | | | | | | | | | |
| | | | | Anaplastic | | | | | | | | | | | |
| | | | | Astrocytoma | | | | | | | | | | | |
| 25 | 50 | M | IV | GBM | WT | Right occipital | 30.41 | YES | 8 | NO | C228T | C228T | 766 | 18.25 | 1991.25 |
| 26 | 38 | M | II | Diffuse | WT | Right frontal | 115.36 | YES | Incidental | NO | WT | WT | 3.5 | 6.75 | 888.75 |
| | | | | Astrocytoma | | | | | Finding | | | | | | |
| 27 | 29 | F | III | Anaplastic | IDH1 | Left temporal | 702.49 | YES | 4 | NO | WT | WT | 2.3 | 5.5 | 852.5 |
| | | | | Astrocytoma | R132H | | | | | | | | | | |
| 28 | 51 | F | III | Anaplastic | WT | Right temporal | 65.6 | YES | 1 | NO | WT | WT | 7.4 | 2.13 | 973.75 |
| | | | | Astrocytoma | | | | | | | | | | | |
| 29 | 50 | M | IV | Recurrent/ | WT | Right frontal | 17.91 | YES | N/A | YES | C250T | C250T | 639 | 10.13 | 853.75 |
| | | | | Residual GBM | | | | | | | | | | | |
| 30 | 69 | F | IV | GBM | WT | Cerebellar | 16.91 | YES | 5 | NO | WT | WT | 7.3 | 7.38 | 2983.75 |
| 31 | 31 | F | IV | GBM | IDH1 | Right frontal | 45.24 | YES | N/A | YES | WT | WT | 0 | 0 | 2083.75 |
| | | | | | R132G | | | | | | | | | | |
| 32 | 48 | F | N/A | N/A | N/A | N/A | N/A | N/A | N/A | N/A | WT | N/A | N/A | 5.75 | 443.75 |
| 33 | 28 | F | N/A | N/A | N/A | N/A | N/A | N/A | N/A | N/A | WT | N/A | N/A | 0 | 671.25 |
| 34 | 24 | F | N/A | N/A | N/A | N/A | N/A | N/A | N/A | N/A | WT | N/A | N/A | 3.25 | 1176.25 |
| 35 | 29 | F | N/A | N/A | N/A | N/A | N/A | N/A | N/A | N/A | WT | N/A | N/A | 0 | 1812.5 |
| 36 | 23 | F | N/A | N/A | N/A | N/A | N/A | N/A | N/A | N/A | WT | N/A | N/A | 0 | 1892.5 |
| 37 | 23 | F | N/A | N/A | N/A | N/A | N/A | N/A | N/A | N/A | WT | N/A | N/A | 1.25 | 567.5 |
| 38 | 25 | F | N/A | N/A | N/A | N/A | N/A | N/A | N/A | N/A | WT | N/A | N/A | 1 | 1023.75 |
| 39 | 33 | M | N/A | N/A | N/A | N/A | N/A | N/A | N/A | N/A | WT | N/A | N/A | 0 | 1753.75 |
| 40 | 30 | F | N/A | N/A | N/A | N/A | N/A | N/A | N/A | N/A | WT | N/A | N/A | 3.5 | 726.25 |
| 41 | 24 | F | N/A | N/A | N/A | N/A | N/A | N/A | N/A | N/A | WT | N/A | N/A | 1.88 | 1230 |
| 42 | 70 | F | IV | GBM | WT | Corpus callosum/ | 102.22 | YES | 3 | NO | C228T | N/A | N/A | 21.13 | 1651.25 |
| | | | | | | lateral ventricles | | | | | | | | | |
| 43 | 71 | M | IV | GBM | WT | Right parietal | 36.88 | YES | 8 | NO | C250T | N/A | N/A | 10.63 | 6325 |
| 44 | 54 | F | IV | GBM | WT | Left thalamic | 176.43 | YES | 2 | NO | C228T | N/A | N/A | 4.25 | 1527.5 |
| 45 | 70 | M | IV | GBM | WT | Left frontal | 423.95 | YES | 4 | NO | C228T | N/A | N/A | 10 | 1587.5 |
| 46 | 59 | F | IV | GBM | WT | Left frontal | 71.43 | YES | N/A | YES | C228T | N/A | N/A | 13.88 | 2441.67 |
| 47 | 69 | M | IV | GBM | WT | Bifrontal, | 37.25 | YES | 3 | NO | C250T | N/A | N/A | 14.125 | 1197.5 |
| | | | | | | corpus callosum | | | | | | | | | |
| 48 | 56 | M | IV | GBM | WT | Left frontal | 88.22 | YES | 4 | NO | C228T | N/A | N/A | 31 | 1203.75 |

TABLE 5
Patient Characteristics for Multi-Institution Cohort. Tumor volume calculated by taking three measurements and using the following formula:
4pi/3 * R1 * R2 * R3. GBM = Glioblastoma, WT = Wildtype, N/A = Not Available, M = Male, F = Female

| | | | | WHO | | | | Volume | Contrast | Duration | | | Mutant Allele |
|---|---|---|---|---|---|---|---|---|---|---|---|---|---|
| Cohort | Study ID | Age | Sex | Grade | Diagnosis | IDH1 Status | Location | (cm\hat{ }3) | Enhancement | (weeks) | Recurrent | TERT Status (MGH) | Frequency |
| Operator | 1820 | 50 | M | IV | GBM w Small primitive | UKN | R Temporal | 106.85 | YES | 2 | NO | C228A | 0.225 |
| | | | | | neuronal component | | | | | | | | |
| Operator | 2083 | 55 | M | III | Anaplastic Astrocytoma | WT | L Occipital | 53.82 | YES | 4 | NO | C228T | 0.609 |
| Multi- | 2416 | 38 | M | III | Anaplastic | IDH1 | L Frontal | 29.4 | YES | N/A | NO | C228T | 2.269289 |
| institutional | | | | | Oligodendroglioma | R132H | | | | | | | |
| Multi- | 2760 | 45 | M | III | Anaplastic | IDH1 | L Frontal | 56.7 | YES | 28 | NO | C228T | 0.32967 |
| institutional | | | | | Oligodendroglioma | R132H | | | | | | | |
| Multi- | 743 | 40 | F | N/A | Astrocytoma with Elevated | WT | optic pathway | 80.69 | YES | 8 | NO | C228T | 0.683761 |
| institutional | | | | | Proliferation Index | | | | | | | | |
| Multi- | 2750 | 68 | M | IV | Diffuse Astrocytic | WT | R Frontoparietal | 16.53 | NO | 6 | NO | C228T | 0.857143 |
| institutional | | | | | Glioma with Molecular | | | | | | | | |
| | | | | | Features of GBM | | | | | | | | |
| Multi- | 2854 | 68 | M | IV | Diffuse Astrocytic | WT | Midline | 83.55 | YES | 16 | NO | C228T | 0.493259 |
| institutional | | | | | Glioma with Molecular | | | | | | | | |
| | | | | | Features of GBM | | | | | | | | |
| Operator | 2341 | 77 | M | IV | Diffuse Astrocytoma | WT | L temporo-occipital | unknown | unknown | 5 | NO | C228T | 0.52 |
| Operator | 79 | 45 | F | II | Diffuse Astrocytoma | WT | L Parietal | 172.65 | NO | 78 | NO | C228T | 2.83 |
| Operator | 1214 | 70 | M | IV | GBM | WT | L Frontal | 134.95 | YES | 4 | NO | C228T | 0.896 |
| Operator | 1784 | 56 | M | IV | GBM | WT | R Temporal | 2.7 | YES | 38 | YES | C228T | 0.949 |
| Operator | 2191 | 41 | M | IV | GBM | WT | R temporal | 85.99 | YES | 3 | NO | C228T | 0.599 |
| Operator | 2208 | 58 | M | IV | GBM | WT | Multifocal; | 103.36 | YES | 5 | NO | C228T | 0.545 |
| | | | | | | | L Frontotemporal | | | | | | |
| Operator | 2230 | 63 | F | IV | GBM | WT | R Temporal | 127.92 | YES | N/A | NO | C228T | 0.657 |
| Multi- | 2279 | 75 | M | IV | GBM | WT | L Frontal | 29.6 | YES | 6 | NO | C228T | 0.458716 |
| institutional | | | | | | | | | | | | | |
| Multi- | 2632 | 72 | M | IV | GBM | WT | L temporal | 29.41 | YES | 1 | NO | C228T | 0.637349 |
| institutional | | | | | | | | | | | | | |
| Operator | 2933 | 54 | M | IV | GBM | WT | R Parieto-occipital | 104.65 | YES | 3 | NO | C228T | 0.766 |
| Operator | 2299 | 50 | M | IV | GBM | WT | R Occipital | N/A | YES | 8 | NO | C228T | 0.906 |
| Multi- | 56 | 65 | M | N/A | Recurrent GBM | Negative | Left Temporal | 29.51 | YES | 247 | YES | C228T | 0.608273 |
| institutional | | | | | | | | | | | | | |
| Multi- | 1983 | 41 | F | N/A | Recurrent/Residual | WT | Occipital | 38.08 | YES | 55 | YES | C228T | 1.856148 |
| institutional | | | | | Diffuse Astrocytoma | | | | | | | | |
| | | | | | with Treatment Effects | | | | | | | | |
| Multi- | 1805 | 52 | F | IV | Recurrent/Residual Glioma | WT | L Temporoparietal | 28.59 | YES | 33 | YES | C228T | 0.248679 |
| institutional | | | | | | | | | | | | | |
| Operator | 1810 | 74 | F | IV | Residual GBM | WT | L Temporal | 5.52 | YES | 3 | NO | C228T | 0.25 |
| Operator | 1799 | 58 | F | IV | GBM | WT | Multifocal; R Thalamic | 129.65 | YES | 0 | NO | C228T | 0.041 |
| Operator | 1218 | 77 | F | III | Anaplastic Astrocytoma | WT | L Fronto-temporal | 100.5 | NO | 24 | NO | C250T | 0.593 |
| Multi- | 2676 | 85 | F | IV | GBM | WT | R Temporal | 47.52 | YES | 3 | NO | C250T | 0.221484 |
| institutional | | | | | | | | | | | | | |
| Multi- | 2309 | 60 | F | IV | GBM | WT | Midline | 29.29 | YES | 59 | YES | C250T | 0.88 |
| institutional | | | | | | | | | | | | | |
| Operator | 1834 | 73 | M | N/A | Recurrent/Residual GBM | WT | L Temporal | 81.18 | YES | 33 | YES | C250T | 1.113 |
| Multi- | 2062 | 56 | F | IV | GBM | WT | L Temporal | Unknown | Unknown | Unknown | NO | MT, Unknown | 2.504817 |
| institutional | | | | | | | | | | | | Variant | |
| Multi- | 2502 | 74 | M | IV | GBM | IDH1 | L Parietal | 106.85 | Unknown | Unknown | NO | MT, Unknown | 2.654867 |
| institutional | | | | | | R132H | | | | | | Variant | |
| Multi- | 2511 | 62 | M | IV | GBM | Negative | R Fronto-parietal | 47.52 | Unknown | Unknown | NO | MT, Unknown | 0.653061 |
| institutional | | | | | | | | | | | | Variant | |
| Multi- | 2521 | 64 | M | IV | GBM | Negative | R Frontal | Unknown | Unknown | Unknown | NO | MT, Unknown | 1.381215 |
| institutional | | | | | | | | | | | | Variant | |
| Multi- | 2535 | 46 | M | IV | GBM | IDH1 | R Parietal | Unknown | Unknown | Unknown | NO | MT, Unknown | 1.473684 |
| institutional | | | | | | R132H | | | | | | Variant | |
| Multi- | 2540 | 48 | M | IV | GBM | Negative | R Parietal | Unknown | Unknown | Unknown | NO | MT, Unknown | 0.565553 |
| institutional | | | | | | | | | | | | Variant | |
| Multi- | 2526 | 60 | F | IV | GBM | WT | L Temporal | Unknown | Unknown | Unknown | YES | MT, Unknown | 0.518135 |
| institutional | | | | | | | | | | | | Variant | |
| Multi- | 2531 | 69 | F | IV | GBM | WT | L Frontal | Unknown | Unknown | Unknown | YES | MT, Unknown | 0.35524 |
| institutional | | | | | | | | | | | | Variant | |
| Multi- | 2492 | 67 | M | IV | Recurrent GBM | WT | R Frontal | Unknown | Unknown | Unknown | YES | MT, Unknown | 0.318269 |
| institutional | | | | | | | | | | | | Variant | |
| Multi- | 2497 | 60 | F | IV | Recurrent GBM | WT | R Occipital | 19.97 | Unknown | Unknown | YES | MT, Unknown | 0.325071 |
| institutional | | | | | | | | | | | | Variant | |
| Multi- | 2545 | 66 | M | IV | Recurrent GBM | WT | R Frontoparietal | 7.58 | Unknown | Unknown | YES | MT, Unknown | 0.30248 |
| institutional | | | | | | | | | | | | Variant | |
| Multi- | 2517 | 62 | M | IV | Recurrent Glio sarcoma with | IDH1 | L Temporal | 29.4 | Unknown | Unknown | YES | MT, Unknown | 2.771619 |
| institutional | | | | | Treatment Related Changes | R132H | | | | | | Variant | |
| Multi- | 2055 | 56 | F | IV | Recurrent/Residual GBM | WT | L Temporoparietal | Unknown | Unknown | Unknown | YES | MT, Unknown | 1.766304 |
| institutional | | | | | | | | | | | | Variant | |
| Multi- | 2514 | 44 | M | N/A | Recurrent/Residual GBM | WT | R parietal | 86 | Unknown | Unknown | YES | MT, Unknown | 0.906149 |
| institutional | | | | | with Treatment | | | | | | | Variant | |
| | | | | | Related Changes | | | | | | | | |
| Operator | 1906 | 31 | M | N/A | Recurrent/Residual | IDH1 | L Parietal | 79.56 | NO | 113 | YES | MT, Unknown | 1.141 |
| | | | | | Oligodendroglioma | R132H | | | | | | Variant | |
| Operator | 3093 | 24 | F | N/A | N/A | N/A | N/A | N/A | N/A | N/A | N/A | N/A | 0.171 |
| Multi- | 3098 | 64 | M | N/A | N/A | N/A | N/A | N/A | N/A | N/A | N/A | N/A | 0.397614 |
| institutional | | | | | | | | | | | | | |
| Operator | 3100 | 85 | M | N/A | N/A | N/A | N/A | N/A | N/A | N/A | N/A | N/A | 0.086 |
| Operator | 3116 | 76 | F | N/A | N/A | N/A | N/A | N/A | N/A | N/A | N/A | N/A | 0.594 |
| Operator & | 3127 | 47 | M | N/A | N/A | N/A | N/A | N/A | N/A | N/A | N/A | N/A | 0.25 |
| Multi- | | | | | | | | | | | | | |
| institutional | | | | | | | | | | | | | |
| Operator | 3129 | 42 | M | N/A | N/A | N/A | N/A | N/A | N/A | N/A | N/A | N/A | 0.384 |
| Multi- | 3135 | 57 | F | N/A | N/A | N/A | N/A | N/A | N/A | N/A | N/A | N/A | 0.178678 |
| institutional | | | | | | | | | | | | | |
| Operator | 3144 | 23 | F | N/A | N/A | N/A | N/A | N/A | N/A | N/A | N/A | N/A | 0.152 |
| Operator | 3150 | 56 | F | N/A | N/A | N/A | N/A | N/A | N/A | N/A | N/A | N/A | 0.457 |
| Multi- | 3162 | 67 | F | N/A | N/A | N/A | N/A | N/A | N/A | N/A | N/A | N/A | 0.43 |
| institutional | | | | | | | | | | | | | |
| Multi- | 3163 | 61 | M | N/A | N/A | N/A | N/A | N/A | N/A | N/A | N/A | N/A | 0.403226 |
| institutional | | | | | | | | | | | | | |
| Multi- | 3170 | 55 | F | N/A | N/A | N/A | N/A | N/A | N/A | N/A | N/A | N/A | 0.32 |
| institutional | | | | | | | | | | | | | |
| Multi- | 3172 | 53 | M | N/A | N/A | N/A | N/A | N/A | N/A | N/A | N/A | N/A | 0.229568 |
| institutional | | | | | | | | | | | | | |
| Multi- | 3229 | 54 | M | N/A | N/A | N/A | N/A | N/A | N/A | N/A | N/A | N/A | 0.310752 |
| institutional | | | | | | | | | | | | | |
| Multi- | 89 | 66 | M | N/A | Non Tumor; | WT | R Frontotemporal | 77.9 | NO | 4 | NO | N/A | 0.104932 |
| institutional | | | | | Biopsy Inconclusive | | | | | | | | |
| Multi- | 2293 | 72 | M | N/A | Non Tumor; Brain parenchyma | WT | R Frontoparietal | 13.23 | YES | 4 | NO | N/A | 0.18 |
| institutional | | | | | with Demyelination, Macrophage | | | | | | | | |
| | | | | | and Lymphocyte-rich Infiltrates, | | | | | | | | |
| | | | | | and Gliosis | | | | | | | | |
| Multi- | 1134 | 55 | M | 0 | Non Tumor; Brain with | N/A | Midline | 8.4 | YES | 0 | NO | N/A | 0 |
| institutional | | | | | Necrosis as well as Reactive | | | | | | | | |
| | | | | | and Inflammatory Changes | | | | | | | | |
| Multi- | 2624 | 44 | F | 0 | Non Tumor; | N/A | L Frontal | 19.49 | YES | 3 | NO | N/A | 0.191113 |
| institutional | | | | | Demylineating Lesion | | | | | | | | |
| Multi- | 2866 | 63 | M | 0 | Non Tumor; Fungal Abscess | N/A | L Frontal | 7.85 | YES | 0 | NO | N/A | 0.42 |
| institutional | | | | | | | | | | | | | |
| Multi- | 2732 | 62 | F | 0 | Non Tumor; Hydrocephalus | N/A | N/A | N/A | N/A | N/A | N/A | N/A | 0.242522 |
| institutional | | | | | | | | | | | | | |
| Multi- | 2821 | 67 | M | 0 | Non Tumor; Normal | N/A | N/A | N/A | N/A | N/A | N/A | N/A | 0.49 |
| institutional | | | | | Pressure Hydrocephalus | | | | | | | | |
| Multi- | 2275 | 46 | M | III | Anaplastic Astrocytoma | IDH1 | L Frontal | 97.24 | NO | 4 | NO | WT | 0 |
| institutional | | | | | | R132H | | | | | | | |
| Operator | 2321 | 29 | F | III | Anaplastic Astrocytoma | IDH1 | L Temporal | 223.61 | NO | 0 | NO | WT | 0.349 |
| | | | | | | R132H | | | | | | | |
| Multi- | 2354 | 51 | F | III | Anaplastic Astrocytoma | WT | R Temporal | 20.88 | YES | 0 | NO | WT | 0.635324 |
| institutional | | | | | | | | | | | | | |
| Multi- | 1818 | 31 | M | III | Anaplastic Astrocytoma | IDHl | R Temporal | 2.7 | NO | 164 | YES | WT | 0.09 |
| institutional | | | | | | R132H | | | | | | | |
| Operator | 2312 | 38 | M | II | Diffuse Astrocytoma | WT | R Frontal | 36.56 | NO | N/A | NO | WT | 0.151 |
| Multi- | 153 | 54 | M | IV | GBM | WT | R Occipital | 7.58 | YES | 3 | NO | WT | 0.24 |
| institutional | | | | | | | | | | | | | |
| Multi- | 2359 | 56 | M | IV | GBM | WT | R Parietal | 19.97 | YES | 3 | NO | WT | 0.719424 |
| institutional | | | | | | | | | | | | | |
| Multi- | 2797 | 66 | M | N/A | Non Tumor; Normal | N/A | N/A | N/A | N/A | N/A | N/A | WT | 0.438917 |
| institutional | | | | | Pressure Hydrocephalus | | | | | | | | |
| Multi- | 1175 | 78 | F | N/A | Non Tumor; Subacute | N/A | R Frontal | 131.67 | YES | 5 | NO | WT | 0.29 |
| institutional | | | | | Intrapenchymal Hemorrhage | | | | | | | | |
| Multi- | 1558 | 53 | F | III | Recurrent/Residual | IDHl | L Frontal | 175.22 | YES | 249 | YES | WT | 0.171024 |
| institutional | | | | | Anaplastic Astrocytoma | R132H | | | | | | | |
| Operator | 1855 | 38 | M | III | Recurrent/Residual | IDHl | Midline | 14.87 | YES | 321 | YES | WT | 0.458 |
| | | | | | Infiltrating Astrocytoma | R132G | | | | | | | |

### Example 4: Detection of TERT Promoter Mutations in cfDNA for Longitudinal Monitoring

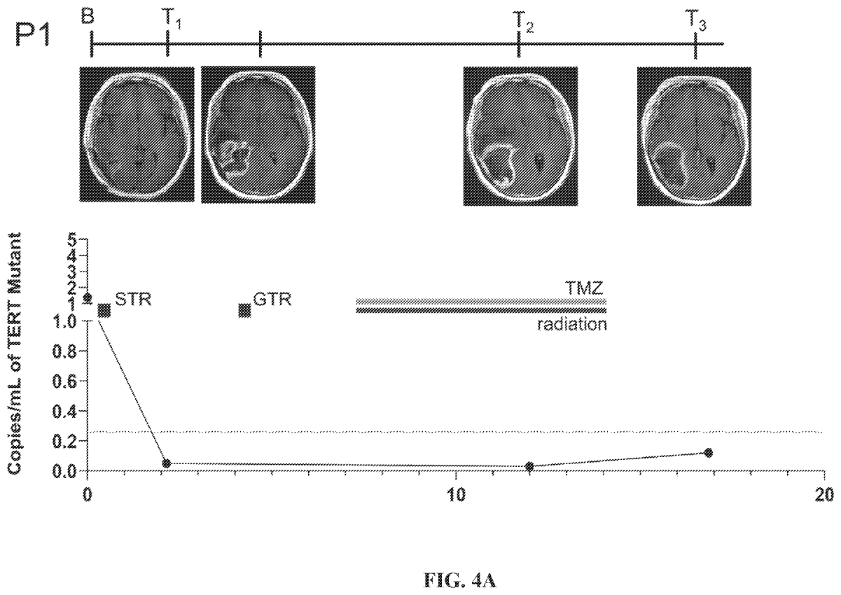

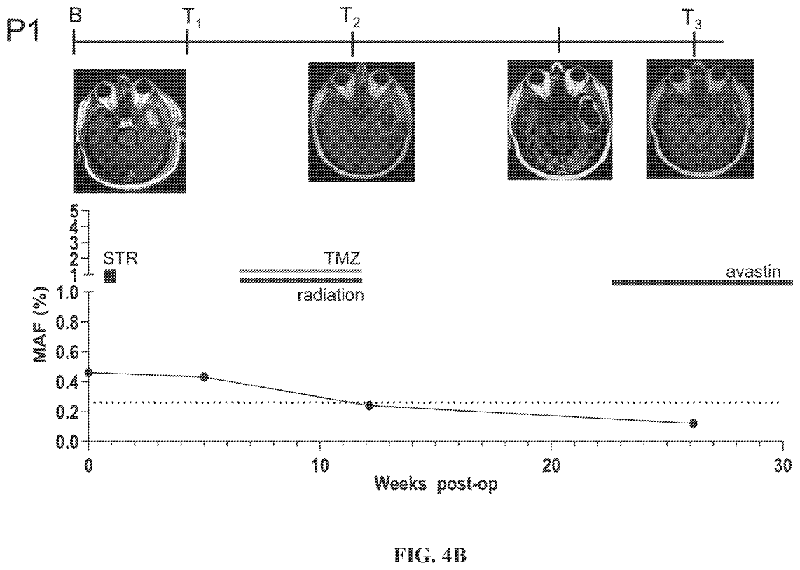

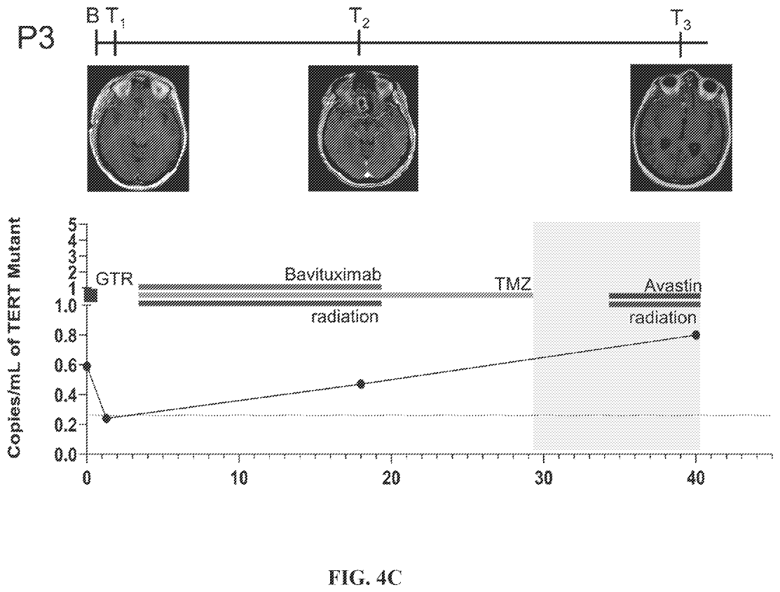

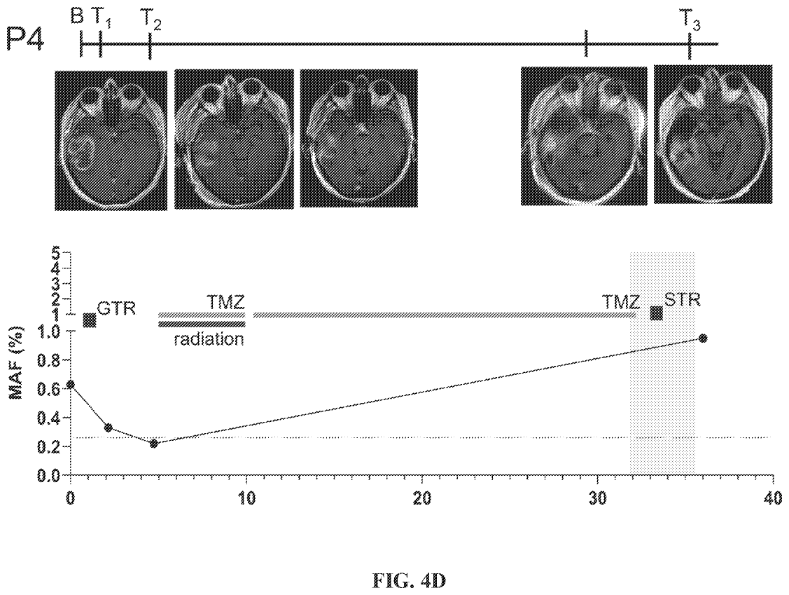

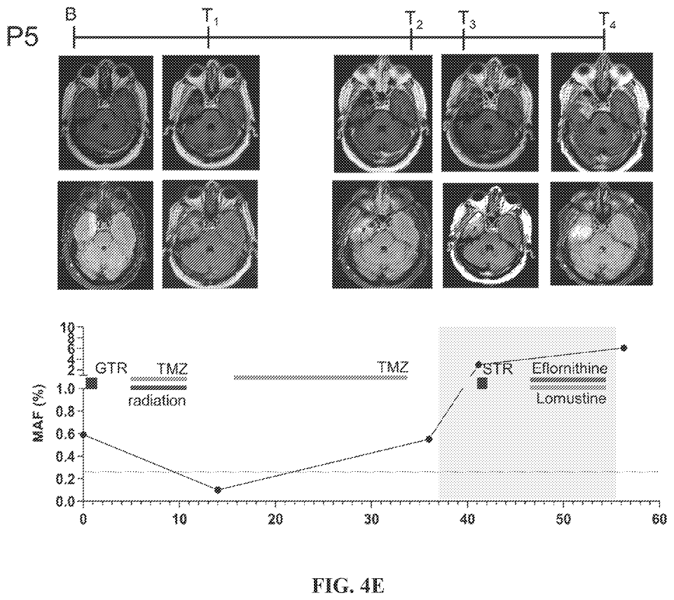

To assess the performance of the TERT assay in a longitudinal setting, cfDNA from serial plasma samples of five patients with TERT mutant glioma was analyzed maintaining the same analytical parameters. TERT mutant copies over the course of therapy paralleled imaging findings and clinical performance of patients (FIGS. 4A-4E). Each of the patients had TERT mutant MAF above threshold prior to initial surgical resection and levels returned to baseline postoperatively. Patients P1 and P2 had stable disease with the TERT mutant levels remaining below baseline over time. On follow up, 3 patients (P3, P4, P5) developed contrast enhancing lesions on MR images after chemoradiation. P4 and P5 had histopathologic confirmation of progression while P3 had clinically diagnosed progression. TERT MAF increased with the development of contrast enhancing recurrent lesions coincident with clinical deterioration. Plasma from P4 was not available for analysis at the time of suspected disease progression.

Taken together, these results demonstrate longitudinal monitoring of TERT promoter mutation in patients with glioma.

### Example 5: Detection of TERT Promoter Mutations in cfDNA from Various Biological Fluids

To assess the performance of the TERT assay using various biological fluids, cfDNA was extracted from 2 mL of cerebrospinal fluid, 2 mL of cleared saliva, and 50 mL of urine using the ExoLution Plus kit (Exosome Diagnostics) according to the manufacturer's instructions. cfDNA was eluted in 20 μL of nuclease free water, and 4 replicates of TERT ddPCR was performed using 4 μL of cfDNA as input. For Urine and Saliva, copies/mL was obtained using the formula Copies/mL=copies (from Quantasoft)*elution volume/template volume/plasma volume. For CSF, all four replicates were merged, and the number of positive Mutant droplets was divided by the number of positive WT droplets to obtain the mutant allele frequency.

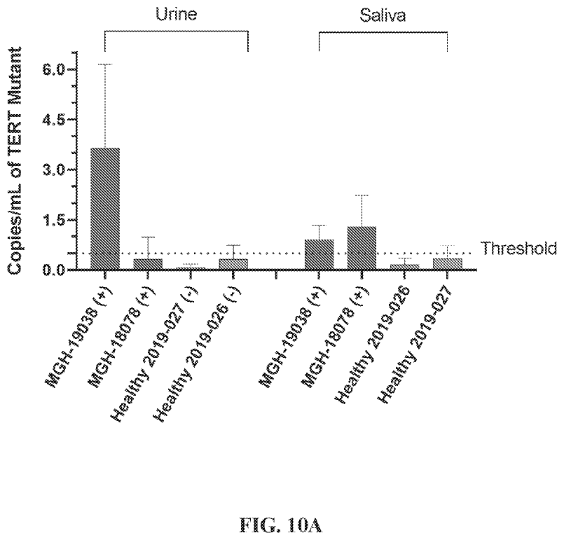

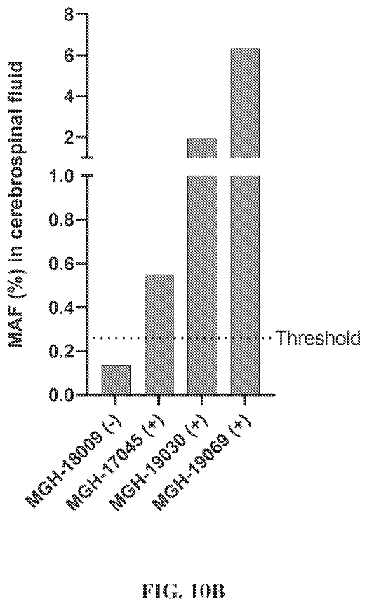

As shown in FIG. 10A, in a small cohort (n=4) of urine samples from patients with TERT mutant gliomas (n=2; indicated with +) and healthy controls (n=2; indicated with −), the TERT assay showed 50% sensitivity and 100% specificity. In testing a similarly small cohort (n=4) of saliva samples from patients with TERT mutant gliomas (n=2; indicated with +) and healthy controls (n=2; indicated with −), the TERT assay showed 100% sensitivity and 100% specificity. As shown in FIG. 10B, in another small cohort (n=4) of CSF samples from patients with TERT mutant gliomas (n=3; indicated with +) and TERT wild-type gliomas (n=1; indicated with −), the TERT assay showed 100% sensitivity and 100% specificity.

Taken together, these results demonstrate that TERT promoter mutations can be detected with high sensitivity and high specificity in various biological fluids from patients.

### Other Embodiments

All of the features disclosed in this specification can be combined in any combination. Each feature disclosed in this specification can be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments can be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases can encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for detecting mutations in a telomerase reverse transcriptase (TERT) promoter sequence, the method comprising:
incubating, in a reaction mixture, a DNA sample comprising the TERT promoter sequence, wherein the DNA sample comprises cell-free DNA (cfDNA) and exosomal nucleic acids (exoNA) extracted from a biological fluid of a subject, a pair of amplification primers comprising a forward primer and a reverse primer, and a pair of detection primers comprising a mutant primer and a wild-type primer, wherein the mutant primer comprises a first detectable label and the wild-type primer comprises a second detectable label, under conditions sufficient for amplifying the TERT promoter sequence, and
detecting a signal from the first detectable label and the second detectable label, wherein presence of the signal from the first detectable label indicates presence of a mutant TERT promoter sequence in the sample and/or wherein presence of the signal from the second detectable label indicates presence of a wild-type TERT promoter sequence in the sample,
wherein the forward primer comprises SEQ ID NO: 1 and the reverse primer comprises SEQ ID NO: 2,
wherein the mutant primer comprises SEQ ID NO: 3 and the wild-type primer comprises SEQ ID NO: 4, and
wherein the mutant TERT promoter sequence comprises C228T or C250T.

2. The method of claim 1, wherein the DNA sample is extracted from the biological fluid using an ExoLution PLUS kit.

3. The method of claim 1 or claim 2, wherein the reaction mixture further comprises 7-deaza-2′-deoxyguanosine 5′-triphosphate (7-deaza-dGTP).

4. The method of any one of claims 1-3, wherein the TERT promoter sequence is amplified by digital PCR (dPCR).

5. The method of claim 4, wherein the TERT promoter sequence is amplified by droplet digital PCR (ddPCR).

6. The method of any one of claims 1-5, wherein the mutant primer comprises at least one locked nucleic acid (LNA) modification and/or wherein the wild-type primer comprises at least one LNA modification.

7. The method of claim 6, wherein the mutant primer comprises LNA modifications at positions 4, 5, 6, and 7 in SEQ ID NO: 3.

8. The method of claim 6 or claim 7, wherein the wild-type primer comprises LNA modifications at positions 5, 6, and 7 in SEQ ID NO: 4.

9. The method of any one of claims 1-8, wherein the forward primer is SEQ ID NO: 1.

10. The method of any one of claims 1-9, wherein the reverse primer is SEQ ID NO: 2.

11. The method of any one of claims 1-10, wherein the mutant primer is SEQ ID NO: 3.

12. The method of any one of claims 1-11, wherein the wild-type primer is SEQ ID NO: 4.

13. The method of any one of claims 1-12, wherein the first detectable label comprises a first fluorophore and a first quencher.

14. The method of any one of claims 1-13, wherein the second detectable label comprises a second fluorophore and a second quencher.

15. The method of claim 13 or claim 14, wherein the first fluorophore and the second fluorophore are different fluorophores.

16. The method of any one of claim 13-15, wherein the first quencher and the second quencher are the same quencher.

17. The method of any one of claims 13-16, wherein the first fluorophore and the second fluorophore are selected from the group consisting of FAM, HEX, Cy3, Cy5, and Texas Red.

18. The method of any one of claims 13-17, wherein the first quencher and the second quencher are selected from the group consisting of Iowa Black FQ, Iowa Black RQ, ZEN Quencher, and TAMRA.

19. The method of any one of claims 1-18, wherein the subject is treatment naïve or wherein the subject has received a cancer therapy.

20. The method of any one of claims 1-19, wherein the biological fluid is selected from the group consisting of plasma, urine, and cerebrospinal fluid (CSF).

21. The method of any one of claims 1-20, wherein the subject is a human patient having or suspected of having a cancer.

22. The method of claim 21, wherein the cancer is selected from brain cancer, skin cancer, lung cancer, liver cancer, breast cancer, thyroid cancer, adrenocortical carcinoma, ovarian cancer, endometrial carcinoma, renal cell carcinoma, bladder cancer, and gastric cancer.

23. The method of claim 22, wherein the brain cancer is a glioma.

24. The method of claim 23, wherein the glioma is selected from the group consisting of an astrocytoma, an ependymoma, and an oligodendroglioma.

25. The method of any one of claims 1-24, further comprising administering a cancer therapy to the subject.

26. The method of claim 25, wherein the cancer therapy is selected from the group consisting of a chemotherapy, a radiation therapy, a surgical therapy, and an immunotherapy.

27. A method of evaluating reoccurrence of a cancer in a subject, the method comprising:
detecting the TERT promoter sequence in the DNA sample from the biological fluid of the subject according to the method of any one of claims 1-31,
determining whether the subject has reoccurrence of the cancer, wherein the subject is identified as having reoccurrence of the cancer when the level of mutant TERT promoter sequences in the sample is higher than a control level, and
administering a cancer therapy to the subject identified as having reoccurrence of the cancer.

28. A method of evaluating effectiveness of a cancer therapy, the method comprising:
detecting the TERT promoter sequence in the DNA sample from the biological fluid of the subject according to the method of any one of claims 1-31,
determining whether the cancer therapy has been effective, wherein the cancer therapy is identified as effective when the level of mutant TERT promoter sequences in the sample is higher than a control level, and
administering the cancer therapy identified as effective to the subject and/or administering another cancer therapy to the subject.

29. An amplification primer for amplifying a promoter region of a telomerase reverse transcriptase (TERT) gene, the amplification primer comprising SEQ ID NO: 2.


SEQUENCE LISTING (5 sequences)

SEQ ID NO: 1 - DNA, 20 bp, Artificial
  TERT gene
cctgccccttcaccttccag

SEQ ID NO: 2 - DNA, 19 bp, Artificial
  TERT gene
agagcggaaaggaagggga

SEQ ID NO: 3 - DNA, 10 bp, Artificial
  primer for TERT gene
  misc_feature (4)..(7): +N modified residue
ccccttccgg

SEQ ID NO: 4 - DNA, 10 bp, Artificial
  primer for TERT gene
  misc_feature (5)..(7): +N modified residue
ccccctccgg

SEQ ID NO: 5 - DNA, 11 bp, Artificial
  template
ccccttccggg